(19) United States
(12) Patent Application Publication
(10) Pub. No.: US 2021/0361704 A1
(43) Pub. Date: Nov. 25, 2021

(54) COMPOSITIONS AND METHODS FOR TCR REPROGRAMMING USING FUSION PROTEINS

(71) Applicant: TCR2 Therapeutics Inc., Cambridge, MA (US)

(72) Inventors: Patrick Alexander BAEUERLE, Gauting (DE); Robert HOFMEISTER, Scituate, MA (US); Daniel GETTS, Westminster, MA (US); Philippe KIEFFER-KWON, Somerville, MA (US); Julie DONAGHEY, Cambridge, MA (US)

(21) Appl. No.: 16/979,380

(22) PCT Filed: Mar. 8, 2019
(86) PCT No.: PCT/US2019/021315

§ 371 Date: Sep. 9, 2020

Related U.S. Application Data
(60) Provisional application No. 62/641,159, filed on Mar. 9, 2018.

(51) Int. Cl. A61K 35/17; C07K 16/28; C07K 14/725; C12N 15/11; A61P 35/00

(52) U.S. Cl. CPC A61K 35/17; C07K 16/2803; A61P 35/00; C12N 15/111; C07K 14/7051

(57) ABSTRACT

Provided herein are recombinant nucleic acids encoding T cell receptor (TCR) fusion proteins (TFPs) and a TCR constant domain, modified T cells expressing the encoded molecules, and methods of use thereof for the treatment of diseases, including cancer.

### CROSS-REFERENCE

This application claims the benefit of U.S. Provisional Patent Application No. 62/641,159, filed Mar. 9, 2018, which is entirely incorporated herein by reference.

### BACKGROUND OF THE INVENTION

Most patients with hematological malignancies or with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue, or are poorly immunogenic. Furthermore, cancer cells use multiple mechanisms to render themselves invisible or hostile to the initiation and propagation of an immune attack by cancer immunotherapies.

Recent developments using chimeric antigen receptor (CAR) modified autologous T cell therapy, which relies on redirecting genetically engineered T cells to a suitable cell-surface molecule on cancer cells, show promising results in harnessing the power of the immune system to treat B cell malignancies (see, e.g., Sadelain et al., Cancer Discovery 3:388-398 (2013)). The clinical results with CD19-specific CAR T cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma.

Besides the ability for genetically modified T cells expressing a CAR or a second TCR to recognize and destroy respective target cells in vitro/ex vivo, successful patient therapy with engineered T cells may require the T cells to be capable of strong activation, expansion, persistence over time, and, in case of relapsing disease, to enable a ‘memory’ response. High and manageable clinical efficacy of CAR T cells is currently limited to CD19-positive B cell malignancies and to NY-ESO-1-peptide expressing synovial sarcoma patients expressing HLA-A2.

### SUMMARY OF THE INVENTION

There is a clear need to improve genetically engineered T cells to more broadly act against various human malignancies.

Described herein are modified T cells comprising fusion proteins of TCR subunits, including CD3 epsilon, CD3gamma, CD3 delta, TCR gamma, TCR delta, TCR alpha and TCR beta chains with binding domains specific for cell surface antigens that have the potential to overcome limitations of existing approaches. Additionally, these modified T cells may have functional disruption of an endogenous TCR (e.g. TCR alpha, beta or both). These modified T cells may have the ability to kill target cells more efficiently than CARs, but release comparable or lower levels of pro-inflammatory cytokines. These modified T cells and methods of their use may represent an advantage for these cells relative to CARs because elevated levels of these cytokines have been associated with dose-limiting toxicities for adoptive CAR-T therapies.

Provided herein are modified T cells comprising T-cell receptor (TCR) fusion protein (TFP) and a TCR constant domain, methods of producing the modified T cells, and methods of use thereof for the treatment of diseases.

Disclosed herein, in some embodiments, are recombinant nucleic acid comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR gamma, TCR delta, TCR alpha or TCR beta, and (ii) a human or humanized antibody comprising an antigen binding domain; and (b) a sequence encoding a TCR constant domain, wherein the TCR constant domain is a TCR alpha constant domain, a TCR beta constant domain, a TCR alpha constant domain and a TCR beta constant domain, a TCR gamma constant domain, a TCR delta constant domain, or a TCR gamma constant domain and a TCR delta constant domain; wherein the TCR subunit and the antibody are operatively linked, and wherein the TFP functionally incorporates into a TCR complex when expressed in a T cell.

Disclosed herein, in some embodiments, are recombinant nucleic acid comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) a binding ligand or a fragment thereof that is capable of binding to an antibody or fragment thereof, and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the binding ligand or fragment thereof are operatively linked, and wherein the TFP functionally incorporates into a TCR complex when expressed in a T cell comprising a functional disruption of an endogenous TCR. In some instances, the binding ligand is capable of binding an Fc domain of the antibody. In some instances, the binding ligand is capable of selectively binding an IgG1 antibody. In some instances, the binding ligand is capable of specifically binding an IgG1 antibody. In some instances, the antibody or fragment thereof binds to a cell surface antigen. In some instances, the antibody or fragment thereof binds to a cell surface antigen on the surface of a tumor cell. In some instances, the binding ligand comprises a monomer, a dimer, a trimer, a tetramer, a pentamer, a hexamer, a heptamer, an octomer, a nonamer, or a decamer. In some instances, the binding ligand does not comprise an antibody or fragment thereof. In some instances, the binding ligand comprises a CD16 polypeptide or fragment thereof. In some instances, the binding ligand comprises a CD16-binding polypeptide. In some instances, the binding ligand is human or humanized. In some instances, the recombinant nucleic acid further comprises a nucleic acid sequence encoding an antibody or fragment thereof capable of being bound by the binding ligand. In some instances, the antibody or fragment thereof is capable of being secreted from a cell.

Disclosed herein, in some embodiments, are recombinant nucleic acid comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) an antigen domain comprising a ligand or a fragment thereof that binds to a receptor or polypeptide expressed on a surface of a cell; and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the antigen domain are operatively linked, and wherein the TFP functionally incorporates into a TCR complex when expressed in a T cell comprising a functional disruption of an endogenous TCR. In some instances, the antigen domain comprises a ligand. In some instances, the ligand binds to the receptor of a cell. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MHC class I-related glycoprotein. In some instances, the MIIC class I-related glycoprotein is selected from the group consisting of MICA, MICB, RAETIE, RAET1G, ULBP1, ULBP2, ULBP3, ULBP4 and combinations thereof. In some instances, the antigen domain comprises a monomer, a dimer, a trimer, a tetramer, a pentamer, a hexamer, a heptamer, an octomer, a nonamer, or a decamer. In some instances, the antigen domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen domain does not comprise an antibody or fragment thereof. In some instances, the antigen domain does not comprise a variable region. In some instances, the antigen domain does not comprise a CDR. In some instances, the ligand or fragment thereof is a Natural Killer Group 2D (NKG2D) ligand or a fragment thereof.

In some embodiments, for the recombinant nucleic acids disclosed above, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules. In some instances, the TCR subunit and the antibody domain, the antigen domain or the binding ligand or fragment thereof are operatively linked by a linker sequence. In some instances, the linker sequence comprises (G₄S)ₙ, wherein n=1 to 4. In some instances, the transmembrane domain is a TCR transmembrane domain from CD3 epsilon, CD3 gamma, CD3 delta, TCR alpha or TCR beta. In some instances, the intracellular domain is derived from only CD3 epsilon, only CD3 gamma, only CD3 delta, only TCR alpha or only TCR beta. In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit. In some instances, the TCR extracellular domain comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. In some instances, the TCR subunit comprises a transmembrane domain comprising a transmembrane domain of a protein selected from the group consisting of a TCR alpha chain, a TCR beta chain, a TCR gamma chain, a TCR delta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications. In some instances, the TCR subunit comprises a TCR intracellular domain comprising a stimulatory domain of a protein selected from an intracellular signaling domain of CD3 epsilon, CD3 gamma or CD3 delta, or an amino acid sequence having at least one modification thereto. In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto. In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto. In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit. In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof, (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof, or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof. In some instances, the at least one but not more than 20 modifications thereto comprise a modification of an amino acid that mediates cell signaling or a modification of an amino acid that is phosphorylated in response to a ligand binding to the TFP. In some instances, the human or humanized antibody is an antibody fragment. In some instances, the antibody fragment is a scFv, a single domain antibody domain, a V_{H} domain or a V_{L} domain. In some instances, human or humanized antibody comprising an antigen binding domain is selected from a group consisting of an anti-CD19 binding domain, anti-B-cell maturation antigen (BCMA) binding domain, anti-mesothelin (MSLN) binding domain, anti-IL13Rα2 binding domain, anti-MUC16 binding domain, anti-CD22 binding domain, anti-PD-1 binding domain, anti-BAFF or BAFF receptor binding domain, and anti-ROR-1 binding domain. In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite. In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid. In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

Disclosed herein, in some embodiments, are vectors comprising the recombinant nucleic acid disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector.

Disclosed herein, in some embodiments, are modified T cell comprising the recombinant nucleic acid disclosed above, or the vector disclosed above; wherein the modified T cell comprises a functional disruption of an endogenous TCR. Further disclosed herein, in some embodiments, are modified T cells comprising the sequence encoding the TFP of the nucleic acid disclosed above or a TFP encoded by the sequence of the nucleic acid disclosed above encoding the TFP, wherein the modified T cell comprises a functional disruption of an endogenous TCR. Also disclosed herein, are modified allogenic T cell comprising the sequence encoding the TFP disclosed above or a TFP encoded by the sequence of the nucleic acid disclosed above encoding the TFP. In some instances, the T cell further comprises a heterologous sequence encoding a TCR constant domain, wherein the TCR constant domain is a TCR alpha constant domain, a TCR beta constant domain or a TCR alpha constant domain and a TCR beta constant domain. In some instances, the endogenous TCR that is functionally disrupted is an endogenous TCR alpha chain, an endogenous TCR beta chain, or an endogenous TCR alpha chain and an endogenous TCR beta chain. In some instances, the endogenous TCR that is functionally disrupted has reduced binding to MHC-peptide complex compared to that of an unmodified control T cell. In some instances, the functional disruption is a disruption of a gene encoding the endogenous TCR. In some instances, the disruption of a gene encoding the endogenous TCR is a removal of a sequence of the gene encoding the endogenous TCR from the genome of a T cell. In some instances, the T cell is a human T cell. In some instances, the T cell is a CD8+ T cell, a CD4+ T cell, a naïve T cell, a memory stem T cell, a central memory T cell, a double negative T cell, an effector memory T cell, an effector T cell, a ThO cell, a TcO cell, a Th1 cell, a Tc1 cell, a Th2 cell, a Tc2 cell, a Th17 cell, a Th22 cell, a gamma delta T cell, a natural killer (NK) cell, a natural killer T (NKT) cell, a hematopoietic stem cell, or a pluripotent stem cell. In some instances, the T cell is a CD8+ or CD4+ T cell. In some instances, the T cell is an allogenic T cell. In some instances, the modified T cells further comprise a nucleic acid encoding an inhibitory molecule that comprises a first polypeptide comprising at least a portion of an inhibitory molecule, associated with a second polypeptide comprising a positive signal from an intracellular signaling domain. In some instances, the inhibitory molecule comprises the first polypeptide comprising at least a portion of PD1 and the second polypeptide comprising a costimulatory domain and primary signaling domain.

Disclosed herein, in some embodiments, are pharmaceutical compositions comprising: (a) the modified T cells of the disclosure; and (b) a pharmaceutically acceptable carrier.

Disclosed herein, in some embodiments, are method of producing the modified T cell of the disclosure, the method comprising (a) disrupting an endogenous TCR gene encoding a TCR alpha chain, a TCR beta chain, or a TCR alpha chain and a TCR beta chain; thereby producing a T cell containing a functional disruption of an endogenous TCR gene; and (b) transducing the T cell containing a functional disruption of an endogenous TCR gene with the recombinant nucleic acid, or the vector disclosed herein. In some instances, disrupting comprises transducing the T cell with a nuclease protein or a nucleic acid sequence encoding a nuclease protein that targets the endogenous gene encoding a TCR alpha chain, a TCR beta chain, or a TCR alpha chain and a TCR beta chain. Further disclosed herein, in some embodiments, are method of producing the modified T cell of the disclosure, the method comprising transducing a T cell containing a functional disruption of an endogenous TCR gene with the recombinant nucleic acid, or the vector disclosed herein. In some instances, the T cell containing a functional disruption of an endogenous TCR gene is a T cell containing a functional disruption of an endogenous TCR gene encoding a TCR alpha chain, a TCR beta chain, or a TCR alpha chain and a TCR beta chain. In some instances, the T cell is a human T cell. In some instances, the T cell containing a functional disruption of an endogenous TCR gene has reduced binding to MHC-peptide complex compared to that of an unmodified control T cell. In some instances, the nuclease is a meganuclease, a zinc-finger nuclease (ZFN), a transcription activator-like effector nuclease (TALEN), a CRISPR/Cas nuclease, or a megaTAL nuclease. In some instances, the sequence comprised by the recombinant nucleic acid or the vector is inserted into the endogenous TCR subunit gene at the cleavage site, and wherein the insertion of the sequence into the endogenous TCR subunit gene functionally disrupts the endogenous TCR subunit. In some instances, the nuclease is a meganuclease. In some instances, the meganuclease comprises a first subunit and a second subunit, wherein the first subunit binds to a first recognition half-site of the recognition sequence, and wherein the second subunit binds to a second recognition half-site of the recognition sequence. In some instances, the meganuclease is a single-chain meganuclease comprising a linker, wherein the linker covalently joins the first subunit and the second subunit.

Disclosed herein, in some embodiments, are method of treating cancer in a subject in need thereof, the method comprising administering to the subject a therapeutically effective amount of the pharmaceutical composition disclosed herein. Also disclosed herein, in some embodiments, are method of treating cancer in a subject in need thereof, the method comprising administering to the subject a pharmaceutical composition comprising (a) a modified T cell produced according to the methods disclosed herein; and (b) a pharmaceutically acceptable carrier. In some instances, the modified T cell is an allogeneic T cell. In some instances, less cytokines are released in the subject compared a subject administered an effective amount of an unmodified control T cell. In some instances, less cytokines are released in the subject compared a subject administered an effective amount of a modified T cell comprising the recombinant nucleic acid disclosed herein, or the vector disclosed herein. In some instances, the method comprises administering the pharmaceutical composition in combination with an agent that increases the efficacy of the pharmaceutical composition. In some instances, the method comprises administering the pharmaceutical composition in combination with an agent that ameliorates one or more side effects associated with the pharmaceutical composition. In some instances, the cancer is a solid cancer, a lymphoma or a leukemia. In some instances, the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

Disclosed herein, in some embodiments, are recombinant nucleic acid, the vector, the modified T cell, or the pharmaceutical composition disclosed herein, for use as a medicament or in the preparation of a medicament.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a sequence alignment between TRBC1 and TRBC2, selected crRNAs are represented by arrows over TRBC1 sequence.

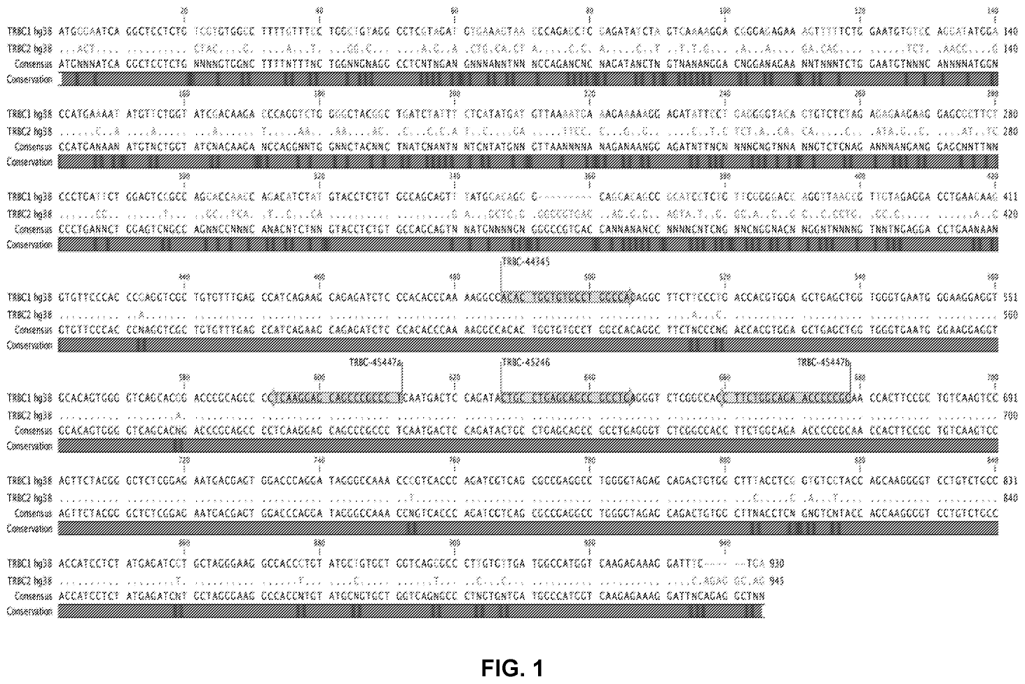

FIGS. 2A-B depict example graphs showing surface expression of CD3 (SK7) vs TCRαβ (IP26) in TRA-edited (FIG. 2A) and TRB-edited (FIG. 2B) cells. Wild type Jurkat cells were edited at either the TRAC or TRBC genes to disrupt TRA or TRB surface expression. Cells negative for CD3 and TCRαβ were purified using Magnetic-Activated Cell Sorting. The gates on the plots were drawn to delineate CD3 and TCRαβ negative-negative population of cells and the percentages of cells remaining in each quadrant are shown in the corners.

FIGS. 3A-E depict example graphs showing surface expression of CD3 vs TCRαβ in wild type cells vs edited (TRA/B disrupted) cells before and after purification. Wild type Donor 1 T cells (FIG. 3A) were edited at either the TRAC (FIG. 3B or FIG. 3D) or TRBC (FIG. 3C or FIG. 3E) genes to disrupt TRA or TRB surface expression. FIG. 3B and FIG. 3C show status of CD3 vs TCRαβ surface markers directly after editing, while FIG. 3D and FIG. 3E show status of these surface markers after their negative selection using Magnetic-Activated Cell Sorting (MACS). The gates on the plots were drawn to delineate CD3 and TCRαβ negative-negative population of cells and the percentages of cells remaining in each quadrant are shown in the corners.

FIG. 4 depicts example graphs measuring the allogenicity of TCR-negative T cells by observing their proliferation rates. TCR-negative T cells were permanently labelled with CSFE dye which halves its concentration with cellular division. Full gray peaks along the X-axis show CSFE signal in unlabeled cells as a negative control. Gray lines show CSFE amount in cells after 24 hours without any stimulation while black lines indicate CSFE amounts after 5 days of co-culture (with stimulation). Y-axis indicates percentage of cells. TRA negative T cells are shown in the top four plots, while TRB negative T cells are shown on the bottom four plots. Allo reaction indicates the TRA KO Donor 2 T cells were mixed with PBMCs from a Donor of a different haplotype (Donor 1), while Auto reaction indicates that T cells and PBMCs of the same Donor were co-cultured. Positive control for TCR independent stimulation was indicated in the PMA and Ionomycin panels.

FIG. 5 depicts example strategies to generate allogeneic TFP T cells. The numbers below correspond to the numbered drawings in FIG. 5. (1) shows endogenous TCRαβ on a T cell interacting with MHCI on an antigen presenting cell and an antigen. (2) shows co-expression of TRBC with TRAC fused to a TFP binder, in TRA−/− or TRB−/− cells. (3) shows co-expression of mouse TRBC with mouse TRAC fused to a TFP binder, in TRA−/− or TRB−/− cells. (4) shows co-expression of murinized TRBC with murinized TRAC fused to a TFP binder, in TRA−/− or TRB−/− cells. (5) shows a TFP binder carried by an enhanced TRAC protein with strong affinity for TCRβ in TRA−/− cells. (6) shows a strategy wherein, in order to enhance the interaction between TRAC and TRBC, the IgG constant domains were fused at the C-terminal end of each of the TCR constant domains. The TFP binder is fused to the C-terminal end of IgG constant domain in TRA−/− or TRB−/− cells. (7) shows a strategy wherein the N-terminal parts of TRAC and TRBC were replaced by their homolog parts in TCRγ and TCRδ, respectfully. The TFP binder is carried by TRAC and/or TRBC in TRA−/− or TRB−/− cells.

FIG. 6 depicts an example schematic showing knock-in strategy of the T2A self-cleaving sequence to enable generation of allogeneic TFP T cells.

FIG. 7 depicts example graphs showing surface expression of TCRαβ and CD3ε (human) or mouse TCRβ as determined by a Luc-Cyto assay as described in Example 6.

FIG. 8 depicts example graphs showing Luc-Cyto analysis of T effector cells cultured with tumor target cells (Nalm 6 cells on the top panel, K562 cells on the bottom panel) at 3-to-1, 1-to-1, or 1-to-3 ratios. Target (CD19 positive) cells are shown in the left panel. The x-axes represent percentage of tumor cell lysis.

FIGS. 9A-C depict example graphs showing surface expression of CD3 vs TCRαβ in wild type cells (FIG. 9A), TRB KO cells without transduction (FIG. 9B), TRB KO cells with transduction of TCRβ full length (FL) TFPs (FIG. 9C), The gates on the plots were drawn to delineate CD3 and TCRαβ negative-negative population of cells and the percentages of cells remaining in each quadrant are shown in the corners.

FIGS. 10A-B depict example graphs showing surface expression of CD3 vs TCRαβ in TRB knockout cells transduced with a human TRBC gene (FIG. 10A) and with a murine TRAC-T2A-TRBC gene (FIG. 10B). The gates on the plots were drawn to delineate CD3 and TCRαβ negative-negative population of cells and the percentages of cells remaining in each quadrant are shown in the corners.

### DETAILED DESCRIPTION OF THE INVENTION

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha, TCR beta, TCR gamma, or TCR delta, and (ii) a human or humanized antibody comprising an antigen binding domain; and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the antibody are operatively linked, and wherein the TFP functionally incorporates into a TCR complex when expressed in a T cell.

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) a binding ligand or a fragment thereof that is capable of binding to an antibody or fragment thereof, and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the binding ligand or fragment thereof are operatively linked, and wherein the TFP functionally incorporates into a TCR complex when expressed in a T cell.

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) an antigen domain comprising a ligand or a fragment thereof that binds to a receptor or polypeptide expressed on a surface of a cell; and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the antigen domain are operatively linked, and wherein the TFP functionally incorporates into a TCR complex when expressed in a T cell.

Disclosed herein, in some embodiments, are vectors comprising the recombinant nucleic acid disclosed herein.

Disclosed herein, in some embodiments, are modified T cells comprising the recombinant nucleic acid disclosed herein, or the vectors disclosed herein; wherein the modified T cell comprises a functional disruption of an endogenous TCR.

Disclosed herein, in some embodiments, are modified T cells comprising the sequence encoding the TFP of the nucleic acid disclosed herein or a TFP encoded by the sequence of the nucleic acid disclosed herein, wherein the modified T cell comprises a functional disruption of an endogenous TCR.

Disclosed herein, in some embodiments, are modified allogenic T cells comprising the sequence encoding the TFP disclosed herein or a TFP encoded by the sequence of the nucleic acid disclosed herein.

Disclosed herein, in some embodiments, are pharmaceutical compositions comprising: (a) the modified T cells of the disclosure; and (b) a pharmaceutically acceptable carrier.

Disclosed herein, in some embodiments, are methods of producing the modified T cell of the disclosure, the method comprising (a) disrupting an endogenous TCR gene encoding a TCR alpha chain, a TCR beta chain, or a TCR alpha chain and a TCR beta chain; thereby producing a T cell containing a functional disruption of an endogenous TCR gene; and (b) transducing the T cell containing a functional disruption of an endogenous TCR gene with the recombinant nucleic acid of the disclosure, or the vectors disclosed herein.

Disclosed herein, in some embodiments, are methods of producing the modified T cell of the disclosure, the method comprising transducing a T cell containing a functional disruption of an endogenous TCR gene with the recombinant nucleic acid disclosed herein, or the vectors disclosed herein.

Disclosed herein, in some embodiments, are methods of treating cancer in a subject in need thereof, the method comprising administering to the subject a therapeutically effective amount of the pharmaceutical compositions disclosed herein.

Disclosed herein, in some embodiments, are methods of treating cancer in a subject in need thereof, the method comprising administering to the subject a pharmaceutical composition comprising (a) a modified T cell produced according to the methods disclosed herein; and (b) a pharmaceutically acceptable carrier.

### Certain Terminology

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g. Lieberman, Pharmaceutical Dosage Forms (vols. 1-3, 1992); Lloyd, The Art, Science and Technology of Pharmaceutical Compounding (1999); and Pickar, Dosage Calculations (1999))

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

“Major histocompatability complex (MHC) molecules are typically bound by TCRs as part of peptide:MHC complex. The MHC molecule may be an MHC class I or II molecule. The complex may be on the surface of an antigen presenting cell, such as a dendritic cell or a B cell, or any other cell, including cancer cells, or it may be immobilized by, for example, coating on to a bead or plate.

The human leukocyte antigen system (HLA) is the name of the gene complex which encodes major histocompatibility complex (MHC) in humans and includes HLA class I antigens (A, B & C) and HLA class II antigens (DP, DQ, & DR). HLA alleles A, B and C present peptides derived mainly from intracellular proteins, e.g., proteins expressed within the cell.

During T cell development in vivo, T cells undergo a positive selection step to ensure recognition of self MHCs followed by a negative step to remove T cells that bind too strongly to MHC which present self-antigens. As a consequence, certain T cells and the TCRs they express will only recognize peptides presented by certain types of MHC molecules—i.e. those encoded by particular HLA alleles. This is known as HLA restriction.

One HLA allele of interest is HLA-A*0201, which is expressed in the vast majority (>50%) of the Caucasian population. Accordingly, TCRs which bind WT1 peptides presented by MHC encoded by HLA-A*0201 (i.e. are HLA-A*0201 restricted) are advantageous since an immunotherapy making use of such TCRs will be suitable for treating a large proportion of the Caucasian population.

Other HLA-A alleles of interest are HLA-A*0101, HLA-A*2402, and HLA-A*0301.

Widely expressed HLA-B alleles of interest are HLA-B*3501, HLA-B*0702 and HLA-B*3502.

An “intracellular signaling domain,” as the term is used herein, refers to an intracellular portion of a molecule. The intracellular signaling domain generates a signal that promotes an immune effector function of the TFP containing cell, e.g., a modified T-T cell. Examples of immune effector function, e.g., in a modified T-T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” refers to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies such as sdAb (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “V_{H}” with regard to an antibody refers to the fragment of the heavy chain that contains three CDRs interposed between flanking stretches known as framework regions, these framework regions are generally more highly conserved than the CDRs and form a scaffold to support the CDRs. A camelid “V_{H}H” domain is a heavy chain comprising a single variable antibody domain.

Unless specified, as used herein a scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb), a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “recombinant antibody” refers to an antibody that is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

As used herein, the term “CD19” refers to the Cluster of Differentiation 19 protein, which is an antigenic determinant detectable on B cell leukemia precursor cells, other malignant B cells and most cells of the normal B cell lineage.

As used herein, the term “BCMA” refers to the B-cell maturation antigen also known as tumor necrosis factor receptor superfamily member 17 (TNFRSF17) and Cluster of Differentiation 269 protein (CD269) is a protein that in humans is encoded by the TNFRSF17 gene. TNFRSF17 is a cell surface receptor of the TNF receptor superfamily which recognizes B-cell activating factor (BAFF) (see, e.g., Laabi et al., EMBO 11 (11): 3897-904 (1992). This receptor is expressed in mature B lymphocytes, and may be important for B-cell development and autoimmune response.

As used herein, the term “CD16” (also known as FcγRIII) refers to a cluster of differentiation molecule found on the surface of natural killer cells, neutrophil polymorphonuclear leukocytes, monocytes and macrophages. CD16 has been identified as Fc receptors FcγRIIIa (CD16a) and FcγRIIIb (CD16b), which participate in signal transduction. CD16 is a molecule of the immunoglobulin superfamily (IgSF) involved in antibody-dependent cellular cytotoxicity (ADCC).

“NKG2D,” as used herein, refers to a transmembrane protein belonging to the CD94/NKG2 family of C-type lectin-like receptors. In humans, NKG2D is expressed by NK cells, γδ T cells and CD8+ αβ T cells. NKG2D recognizes induced-self proteins from MIC and RAET1/ULBP families which appear on the surface of stressed, malignant transformed, and infected cells.

Mesothelin (MSLN) refers to a tumor differentiation antigen that is normally present on the mesothelial cells lining the pleura, peritoneum and pericardium. Mesothelin is over expressed in several human tumors, including mesothelioma and ovarian and pancreatic adenocarcinoma.

Tyrosine-protein kinase transmembrane receptor ROR1, also known as neurotrophic tyrosine kinase, receptor-related 1 (NTRKR1) is a member of the receptor tyrosine kinase-like orphan receptor (ROR) family. It plays a role in metastasis of cancer.

The term “MUC16”, also known as “mucin 16, cell-surface associated” or “ovarian cancer-related tumor marker CA125” is a membrane-tethered mucin that contains an extracellular domain at its amino terminus, a large tandem repeat domain, and a transmembrane domain with a short cytoplasmic domain. Products of this gene have been used as a marker for different cancers, with higher expression levels associated with poorer outcomes.

The term “CD22,” also known as sialic acid binding Ig-like lectin 2, SIGLEC-2, T cell surface antigen leu-14, and B cell receptor CD22, is a protein that mediates B cell/B cell interactions, and is thought to be involved in the localization of B cells in lymphoid tissues, and is associated with diseases including refractory hematologic cancer and hairy cell leukemia. A fully human anti-CD22 monoclonal antibody (“M971”) suitable for use with the methods disclosed herein is described, e.g., in Xiao et al., MAbs. 2009 May-June; 1(3): 297-303.

The “CD79α” and “CD79β” genes encode proteins that make up the B lymphocyte antigen receptor, a multimeric complex that includes the antigen-specific component, surface immunoglobulin (Ig). Surface Ig non-covalently associates with two other proteins, Ig-alpha and Ig-beta (encoded by CD79α and its paralog CD79β, respectively) which are necessary for expression and function of the B-cell antigen receptor. Functional disruption of this complex can lead to, e.g., human B-cell chronic lymphocytic leukemias.

B cell activating factor, or “BAFF” is a cytokine that belongs to the tumor necrosis factor (TNF) ligand family. This cytokine is a ligand for receptors TNFRSF13B/TACI, TNFRSF17/BCMA, and TNFRSF13C/BAFF-R. This cytokine is expressed in B cell lineage cells, and acts as a potent B cell activator. It has been also shown to play an important role in the proliferation and differentiation of B cells.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

The term “autologous” refers to any material derived from the same individual to whom it is later to be re-introduced into the individual.

The term “allogeneic” or, alternatively, “allogenic,” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “functional disruption” refers to a physical or biochemical change to a specific (e.g., target) nucleic acid (e.g., gene, RNA transcript, of protein encoded thereby) that prevents its normal expression and/or behavior in the cell. In one embodiment, a functional disruption refers to a modification of the gene via a gene editing method. In one embodiment, a functional disruption prevents expression of a target gene (e.g., an endogenous gene).

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the present disclosure can be replaced with other amino acid residues from the same side chain family and the altered TFP can be tested using the functional assays described herein.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the present disclosure are linkers described in WO2012/138475 (incorporated herein by reference). In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, lung cancer, liver cancer, NHL, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., CD19) present in a sample, but which does not necessarily and substantially recognize or bind other molecules in the sample.

As used herein, the term “meganuclease” refers to an endonuclease that binds double-stranded DNA at a recognition sequence that is greater than 12 base pairs. Preferably, the recognition sequence for a meganuclease of the present disclosure is 22 base pairs. A meganuclease can be an endonuclease that is derived from I-Crel and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art (e.g., WO 2007/047859). A meganuclease as used herein binds to double-stranded DNA as a heterodimer or as a “single-chain meganuclease” in which a pair of DNA-binding domains are joined into a single polypeptide using a peptide linker. The term “homing endonuclease” is synonymous with the term “meganuclease.” Meganucleases of the present disclosure are substantially non-toxic when expressed in cells, particularly in human T cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “TALEN” refers to an endonuclease comprising a DNA-binding domain comprising 16-22 TAL domain repeats fused to any portion of the Fokl nuclease domain.

As used herein, the term “Compact TALEN” refers to an endonuclease comprising a DNA-binding domain with 16-22 TAL domain repeats fused in any orientation to any catalytically active portion of nuclease domain of the I-Tev1 homing endonuclease.

As used herein, the term “CRISPR” refers to a caspase-based endonuclease comprising a caspase, such as Cas9, and a guide RNA that directs DNA cleavage of the caspase by hybridizing to a recognition site in the genomic DNA.

As used herein, the term “megaTAL” refers to a single-chain nuclease comprising a transcription activator-like effector (TALE) DNA binding domain with an engineered, sequence-specific homing endonuclease.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### DESCRIPTION

Provided herein are compositions of matter and methods of use for the treatment of a disease such as cancer, using modified T cells comprisisng a T cell receptors (TCR) fusion protein (TFP and a TCR constant domain, wherein the modified T cell also has a functionally disrupted endogenous TCR subunit. As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. As provided herein, TFPs provide substantial benefits as compared to Chimeric Antigen Receptors. The term “Chimeric Antigen Receptor” or alternatively a “CAR” refers to a recombinant polypeptide comprising an extracellular antigen binding domain in the form of a scFv, a transmembrane domain, and cytoplasmic signaling domains (also referred to herein as “an intracellular signaling domains”) comprising a functional signaling domain derived from a stimulatory molecule as defined below. Generally, the central intracellular signaling domain of a CAR is derived from the CD3 zeta chain that is normally found associated with the TCR complex. The CD3 zeta signaling domain can be fused with one or more functional signaling domains derived from at least one co-stimulatory molecule such as 4-1BB (i.e., CD137), CD27 and/or CD28.

### T Cell Receptor (TCR) Fusion Proteins (TFP)

The present disclosure encompasses recombinant DNA constructs encoding TFPs, wherein the TFP comprises an antibody fragment that binds specifically to CD19, e.g., human CD19, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to BCMA, e.g., human BCMA, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to ROR1, e.g., human ROR1, wherein the sequence of the antibody fragment is contiguous with and in the same reading frame as a nucleic acid sequence encoding a TCR subunit or portion thereof. The present disclosure encompasses recombinant DNA constructs encoding TFPs, wherein the TFP comprises an antibody fragment that binds specifically to CD22, e.g., human CD22, wherein the sequence of the antibody fragment is contiguous with and in the same reading frame as a nucleic acid sequence encoding a TCR subunit or portion thereof. The TFPs provided herein are able to associate with one or more endogenous (or alternatively, one or more exogenous, or a combination of endogenous and exogenous) TCR subunits in order to form a functional TCR complex.

In one aspect, the TFP of the present disclosure comprises a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus examples of cell surface markers that may act as target antigens for the antigen binding domain in a TFP of the present disclosure include those associated with viral, bacterial and parasitic infections; autoimmune diseases; and cancerous diseases (e.g., malignant diseases).

In one aspect, the TFP-mediated T cell response can be directed to an antigen of interest by way of engineering an antigen-binding domain into the TFP that specifically binds a desired antigen.

In one aspect, the portion of the TFP comprising the antigen binding domain comprises an antigen binding domain that targets CD19. In one aspect, the antigen binding domain targets human CD19. In one aspect, the portion of the TFP comprising the antigen binding domain comprises an antigen binding domain that targets BCMA. In one aspect, the antigen binding domain targets human BCMA.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, in one aspect, the antigen-binding domain comprises a humanized or human antibody or an antibody fragment, or a murine antibody or antibody fragment. In one embodiment, the humanized or human anti-CD19 or anti-BCMA binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD19 or anti-BCMA binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD19 binding domain described herein, e.g., a humanized or human anti-CD19 or anti-BCMA binding domain comprising one or more, e.g., all three, LC CDRs and one or more, e.g., all three, HC CDRs. In one embodiment, the humanized or human anti-CD19 binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD19 or anti-BCMA binding domain described herein, e.g., the humanized or human anti-CD19 or anti-BCMA binding domain has two variable heavy chain regions, each comprising a HC CDR1, a HC CDR2 and a HC CDR3 described herein. In one embodiment, the humanized or human anti-CD19 or anti-BCMA binding domain comprises a humanized or human light chain variable region described herein and/or a humanized or human heavy chain variable region described herein. In one embodiment, the humanized or human anti-CD19 or anti-BCMA binding domain comprises a humanized heavy chain variable region described herein, e.g., at least two humanized or human heavy chain variable regions described herein. In one embodiment, the anti-CD19 or anti-BCMA binding domain is a scFv comprising a light chain and a heavy chain of an amino acid sequence provided herein. In an embodiment, the anti-CD19 or anti-BCMA binding domain (e.g., a scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. In one embodiment, the humanized or human anti-CD19 or anti-BCMA binding domain is a scFv, and a light chain variable region comprising an amino acid sequence described herein, is attached to a heavy chain variable region comprising an amino acid sequence described herein, via a linker, e.g., a linker described herein. In one embodiment, the humanized anti-CD19 or anti-BCMA binding domain includes a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a V_{H}4-4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3-1.25 germline sequence. In one embodiment, the framework region can comprise, one, two, three, four or five modifications, e.g., substitutions, e.g., from the amino acid at the corresponding murine sequence.

In some aspects, the portion of a TFP composition of the present disclosure that comprises an antibody fragment is humanized with retention of high affinity for the target antigen and other favorable biological properties. According to one aspect of the present disclosure, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human CD19. In some embodiments, a humanized antibody or antibody fragment may have improved affinity and/or specificity of binding to human CD19 or human BCMA.

In one aspect, the anti-CD19 or anti-BCMA binding domain is characterized by particular functional features or properties of an antibody or antibody fragment. For example, in one aspect, the portion of a TFP composition of the present disclosure that comprises an antigen binding domain specifically binds human CD19 pr human BCMA. In one aspect, the antigen binding domain has the same or a similar binding specificity to human CD19 as the FMC63 scFv described in Nicholson et al. Mol. Immun. 34 (16-17): 1157-1165 (1997). In one aspect, the present disclosure relates to an antigen binding domain comprising an antibody or antibody fragment, wherein the antibody binding domain specifically binds to a CD19 or BCMA protein or fragment thereof, wherein the antibody or antibody fragment comprises a variable light chain and/or a variable heavy chain that includes an amino acid sequence provided herein. In certain aspects, the scFv is contiguous with and in the same reading frame as a leader sequence.

In one aspect, the anti-CD19 or anti-BCMA binding domain is a fragment, e.g., a single chain variable fragment (scFv). In one aspect, the anti-CD19 binding domain is a Fv, a Fab, a (Fab′)₂, or a bi-functional (e.g. bi-specific) hybrid antibody (e.g., Lanzavecchia et al., Eur. J. Immunol. 17, 105 (1987)). In one aspect, the antibodies and fragments thereof of the present disclosure binds a CD19 protein with wild-type or enhanced affinity.

Also provided herein are methods for obtaining an antibody antigen binding domain specific for a target antigen (e.g., CD19, BCMA or any target antigen described elsewhere herein for targets of fusion moiety binding domains), the method comprising providing by way of addition, deletion, substitution or insertion of one or more amino acids in the amino acid sequence of a V_{H} domain set out herein a V_{H} domain which is an amino acid sequence variant of the V_{H} domain, optionally combining the V_{H} domain thus provided with one or more V_{L} domains, and testing the V_{H} domain or V_{H}/V_{L} combination or combinations to identify a specific binding member or an antibody antigen binding domain specific for a target antigen of interest (e.g., CD19 or BCMA) and optionally with one or more desired properties.

In some instances, V_{H} domains and scFvs can be prepared according to method known in the art (see, for example, Bird et al., (1988) Science 242:423-426 and Huston et al., (1988) Proc. Natl. Acad. Sci. USA 85:5879-5883). scFv molecules can be produced by linking V_{H} and V_{L} regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intra-chain folding is prevented. Inter-chain folding is also required to bring the two variable regions together to form a functional epitope binding site. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

A scFv can comprise a linker of about 10, 11, 12, 13, 14, 15 or greater than 15 residues between its V_{L} and V_{H} regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)ₙ, where n is a positive integer equal to or greater than 1. In one embodiment, the linker can be (Gly₄Ser)₄ or (Gly₄Ser)₃. Variation in the linker length may retain or enhance activity, giving rise to superior efficacy in activity studies. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

### Stability and Mutations

The stability of an anti-CD19 or anti-BCMA binding domain, e.g., scFv molecules (e.g., soluble scFv) can be evaluated in reference to the biophysical properties (e.g., thermal stability) of a conventional control scFv molecule or a full length antibody. In one embodiment, the humanized or human scFv has a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent scFv in the described assays.

The improved thermal stability of the anti-CD19 or anti-BCMA binding domain, e.g., scFv is subsequently conferred to the entire CD19-TFP construct, leading to improved therapeutic properties of the anti-CD19 or anti-BCMA TFP construct. The thermal stability of the anti-CD19 or anti-BCMA binding domain, e.g., scFv can be improved by at least about 2° C. or 3° C. as compared to a conventional antibody. In one embodiment, the anti-CD19 or anti-BCMA binding domain, e.g., scFv has a 1° C. improved thermal stability as compared to a conventional antibody. In another embodiment, the anti-CD19 binding domain, e.g., scFv has a 2° C. improved thermal stability as compared to a conventional antibody. In another embodiment, the scFv has a 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the scFv molecules disclosed herein and scFv molecules or Fab fragments of an antibody from which the scFv V_{H} and V_{L} were derived. Thermal stability can be measured using methods known in the art. For example, in one embodiment, T_{M} can be measured. Methods for measuring T_{M} and other methods of determining protein stability are described in more detail below.

Mutations in scFv (arising through humanization or direct mutagenesis of the soluble scFv) alter the stability of the scFv and improve the overall stability of the scFv and the anti-CD19 or anti-BCMA TFP construct. Stability of the humanized scFv is compared against the murine scFv using measurements such as T_{M}, temperature denaturation and temperature aggregation. In one embodiment, the anti-CD19 or anti-BCMA binding domain, e.g., a scFv, comprises at least one mutation arising from the humanization process such that the mutated scFv confers improved stability to the Anti-CD19 TFP construct. In another embodiment, the anti-CD19 binding domain, e.g., scFv comprises at least 1, 2, 3, 4, 5, 6, 7, 8, 9, 10 mutations arising from the humanization process such that the mutated scFv confers improved stability to the CD19-TFP or BCMA-TFP construct.

In one aspect, the antigen binding domain of the TFP comprises an amino acid sequence that is homologous to an antigen binding domain amino acid sequence described herein, and the antigen binding domain retains the desired functional properties of the anti-CD19 or anti-BCMA antibody fragments described herein. In one specific aspect, the TFP composition of the present disclosure comprises an antibody fragment. In a further aspect, that antibody fragment comprises a scFv.

In various aspects, the antigen binding domain of the TFP is engineered by modifying one or more amino acids within one or both variable regions (e.g., V_{H} and/or V_{L}), for example within one or more CDR regions and/or within one or more framework regions. In one specific aspect, the TFP composition of the present disclosure comprises an antibody fragment. In a further aspect, that antibody fragment comprises a scFv.

It will be understood by one of ordinary skill in the art that the antibody or antibody fragment of the present disclosure may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

In one aspect, the present disclosure contemplates modifications of the starting antibody or fragment (e.g., scFv) amino acid sequence that generate functionally equivalent molecules. For example, the V_{H} or V_{L} of an anti-CD19 or anti-BCMA binding domain, e.g., scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{H} or V_{L} framework region of the anti-CD19 binding domain, e.g., scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. In one aspect the extracellular domain is capable of associating with the transmembrane domain. An extracellular domain of particular use in this present disclosure may include at least the extracellular region(s) of e.g., the alpha, beta or zeta chain of the T cell receptor, or CD3 epsilon, CD3 gamma, or CD3 delta, or in alternative embodiments, CD28, CD45, CD4, CD5, CD8, CD9, CD16, CD22, CD33, CD37, CD64, CD80, CD86, CD134, CD137, CD154.

### Transmembrane Domain

In general, a TFP sequence contains an extracellular domain and a transmembrane domain encoded by a single genomic sequence. In alternative embodiments, a TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region. In one aspect, the transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another TFP on the TFP-T cell surface. In a different aspect the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the TFP has bound to a target. A transmembrane domain of particular use in this present disclosure may include at least the transmembrane region(s) of e.g., the alpha, beta, gamma, delta, or zeta chain of the T cell receptor, CD28, CD3 epsilon, CD45, CD4, CD5, CD8, CD9, CD16, CD22, CD33, CD37, CD64, CD80, CD86, CD134, CD137, CD154.

In some instances, the transmembrane domain can be attached to the extracellular region of the TFP, e.g., the antigen binding domain of the TFP, via a hinge, e.g., a hinge from a human protein. For example, in one embodiment, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. In some cases, the linker may be at least about 5, 6, 7, 8, 9, 10, 11, 12, 13, 14, 15, 16, 17, 18, 19, 20, or more in length. A glycine-serine doublet provides a particularly suitable linker. For example, in one aspect, the linker comprises the amino acid sequence of GGGGSGGGGS (SEQ ID NO: 3). In some embodiments, the linker is encoded by a nucleotide sequence of GGTGGCGGAGGTTCTGGAGGTGGAGGTTCC (SEQ ID NO: 4).

### Cytoplasmic Domain

The cytoplasmic domain of the TFP can include an intracellular signaling domain, if the TFP contains CD3 gamma, delta or epsilon polypeptides; TCR alpha and TCR beta subunits are generally lacking in a signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the present disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of naive T cells and that a secondary and/or costimulatory signal is required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the present disclosure include those of CD3 zeta, FcR gamma, FcR beta, CD3 gamma, CD3 delta, CD3 epsilon, CD5, CD22, CD79α, CD79b, and CD66d. In one embodiment, a TFP of the present disclosure comprises an intracellular signaling domain, e.g., a primary signaling domain of CD3-epsilon. In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

The intracellular signaling domain of the TFP can comprise the CD3 zeta signaling domain by itself or it can be combined with any other desired intracellular signaling domain(s) useful in the context of a TFP of the present disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the present disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

In one embodiment, a glycine-serine doublet can be used as a suitable linker. In one embodiment, a single amino acid, e.g., an alanine, a glycine, can be used as a suitable linker.

In one aspect, the TFP-expressing cell described herein can further comprise a second TFP, e.g., a second TFP that includes a different antigen binding domain, e.g., to the same target (CD19 or BCMA) or a different target (e.g., CD123). In one embodiment, when the TFP-expressing cell comprises two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not form an association with the antigen binding domain of the second TFP, e.g., the antigen binding domain of the second TFP is a V_{HH}.

In another aspect, the TFP-expressing cell described herein can further express another agent, e.g., an agent which enhances the activity of a modified T cell. For example, in one embodiment, the agent can be an agent which inhibits an inhibitory molecule. Inhibitory molecules, e.g., PD1, can, in some embodiments, decrease the ability of a modified T cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2, have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). In one embodiment, the PD1 TFP, when used in combinations with an anti-CD19 TFP described herein, improves the persistence of the T cell. In one embodiment, the TFP is a PD1 TFP comprising the extracellular domain of PD 1. Alternatively, provided are TFPs containing an antibody or antibody fragment such as a scFv that specifically binds to the Programmed Death-Ligand 1 (PD-L1) or Programmed Death-Ligand 2 (PD-L2).

In another aspect, the present disclosure provides a population of TFP-expressing T cells, e.g., TFP-T cells. In some embodiments, the population of TFP-expressing T cells comprises a mixture of cells expressing different TFPs. For example, in one embodiment, the population of TFP-T cells can include a first cell expressing a TFP having an anti-CD19 or anti-BCMA binding domain described herein, and a second cell expressing a TFP having a different anti-CD19 or anti-BCMA binding domain, e.g., an anti-CD19 or anti-BCMA binding domain described herein that differs from the anti-CD19 binding domain in the TFP expressed by the first cell. As another example, the population of TFP-expressing cells can include a first cell expressing a TFP that includes an anti-CD19 or anti-BCMA binding domain, e.g., as described herein, and a second cell expressing a TFP that includes an antigen binding domain to a target other than CD19 or BCMA (e.g., another tumor-associated antigen).

In another aspect, the present disclosure provides a population of cells wherein at least one cell in the population expresses a TFP having an anti-CD19 or anti-BCMA domain described herein, and a second cell expressing another agent, e.g., an agent which enhances the activity of a modified T cell. For example, in one embodiment, the agent can be an agent which inhibits an inhibitory molecule. Inhibitory molecules, e.g., can, in some embodiments, decrease the ability of a modified T cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. In one embodiment, the agent that inhibits an inhibitory molecule comprises a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present disclosure also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. In one aspect, the template includes sequences for the TFP.

In one aspect the anti-CD19 or anti-BCMA TFP is encoded by a messenger RNA (mRNA). In one aspect the mRNA encoding the anti-CD19 or anti-BCMA TFP is introduced into a T cell for production of a TFP-T cell. In one embodiment, the in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present disclosure. In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts but do not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one preferred embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However, polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps can also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector®-II (Amaxa Biosystems, Cologne, Germany)), ECM 830 (BTX) (Harvard Instruments, Boston, Mass.) or the Gene Pulser® II (BioRad, Denver, Colo.), Multiporator® (Eppendorf, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Recombinant Nucleic Acid Encoding a TFP and a TCR Constant Domain

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) a human or humanized antibody comprising an antigen binding domain; and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the antibody are operatively linked, and wherein the TFP functionally incorporates into a TCR complex when expressed in a T cell.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the antibody domain, the antigen domain or the binding ligand or fragment thereof are operatively linked by a linker sequence. In some instances, the linker sequence comprises (G4S)n, wherein n=1 to 4.

In some instances, the transmembrane domain is a TCR transmembrane domain from CD3 epsilon, CD3 gamma, CD3 delta, TCR alpha or TCR beta. In some instances, the intracellular domain is derived from only CD3 epsilon, only CD3 gamma, only CD3 delta, only TCR alpha or only TCR beta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain comprising a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79α, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof, (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof, or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-PD-1 binding domain, anti-BAFF or BAFF receptor binding domain, and anti-ROR-1 binding domain.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) a binding ligand or a fragment thereof that is capable of binding to an antibody or fragment thereof, and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the binding ligand or fragment thereof are operatively linked, and wherein the TFP functionally incorporates into a TCR complex when expressed in a T cell. In some instances, the binding ligand is capable of binding an Fc domain of the antibody. In some instances, the binding ligand is capable of selectively binding an IgG1 antibody. In some instances, the binding ligand is capable of specifically binding an IgG1 antibody. In some instances, the antibody or fragment thereof binds to a cell surface antigen. In some instances, the antibody or fragment thereof binds to a cell surface antigen on the surface of a tumor cell. In some instances, the binding ligand comprises a monomer, a dimer, a trimer, a tetramer, a pentamer, a hexamer, a heptamer, an octomer, a nonamer, or a decamer. In some instances, the binding ligand does not comprise an antibody or fragment thereof. In some instances, the binding ligand comprises a CD16 polypeptide or fragment thereof. In some instances, the binding ligand comprises a CD16-binding polypeptide. In some instances, the binding ligand is human or humanized. In some instances, the recombinant nucleic acid further comprises a nucleic acid sequence encoding an antibody or fragment thereof capable of being bound by the binding ligand. In some instances, the antibody or fragment thereof is capable of being secreted from a cell.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the antibody domain, the antigen domain or the binding ligand or fragment thereof are operatively linked by a linker sequence. In some instances, the linker sequence comprises (G₄S)n, wherein n=1 to 4.

In some instances, the transmembrane domain is a TCR transmembrane domain from CD3 epsilon, CD3 gamma, CD3 delta, TCR alpha or TCR beta. In some instances, the intracellular domain is derived from only CD3 epsilon, only CD3 gamma, only CD3 delta, only TCR alpha or only TCR beta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain comprising a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79α, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof, (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof, or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-PD-1 binding domain, anti-BAFF binding domain, and anti-ROR-1 binding domain.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain. Alternatively, the recombinant nucleic acid comprises a sequence encoding a TCR gamma or TCR delta domain, e.g., a transmembrane domain.

Disclosed herein, in some embodiments, are recombinant nucleic acids comprising (a) a sequence encoding a T cell receptor (TCR) fusion protein (TFP) comprising (i) a TCR subunit comprising (1) at least a portion of a TCR extracellular domain, (2) a transmembrane domain, and (3) an intracellular domain comprising a stimulatory domain from an intracellular signaling domain of CD3 epsilon, CD3 gamma, CD3 delta, TCR alpha or TCR beta, and (ii) an antigen domain comprising a ligand or a fragment thereof that binds to a receptor or polypeptide expressed on a surface of a cell; and (b) a sequence encoding a TCR constant domain, wherein the TCR constant domain is a TCR alpha constant domain, a TCR beta constant domain or a TCR alpha constant domain and a TCR beta constant domain; wherein the TCR subunit and the antigen domain are operatively linked, and wherein the TFP functionally incorporates into a TCR complex when expressed in a T cell. In some instances, the antigen domain comprises a ligand. In some instances, the ligand binds to the receptor of a cell. In some instances, the ligand binds to the polypeptide expressed on a surface of a cell. In some instances, the receptor or polypeptide expressed on a surface of a cell comprises a stress response receptor or polypeptide. In some instances, the receptor or polypeptide expressed on a surface of a cell is an MIIC class I-related glycoprotein. In some instances, the MIIC class I-related glycoprotein is selected from the group consisting of MICA, MICB, RAETIE, RAETIG, ULBP1, ULBP2, ULBP3, ULBP4 and combinations thereof. In some instances, the antigen domain comprises a monomer, a dimer, a trimer, a tetramer, a pentamer, a hexamer, a heptamer, an octomer, a nonamer, or a decamer. In some instances, the antigen domain comprises a monomer or a dimer of the ligand or fragment thereof. In some instances, the ligand or fragment thereof is a monomer, a dimer, a trimer, a tetramer, a pentamer, a hexamer, a heptamer, an octomer, a nonamer, or a decamer. In some instances, the ligand or fragment thereof is a monomer or a dimer. In some instances, the antigen domain does not comprise an antibody or fragment thereof. In some instances, the antigen domain does not comprise a variable region. In some instances, the antigen domain does not comprise a CDR. In some instances, the ligand or fragment thereof is a Natural Killer Group 2D (NKG2D) ligand or a fragment thereof.

In some instances, the TCR constant domain incorporates into a functional TCR complex when expressed in a T cell. In some instances, the TCR constant domain incorporates into a same functional TCR complex as the functional TCR complex that incorporates the TFP when expressed in a T cell. In some instances, the sequence encoding the TFP and the sequence encoding the TCR constant domain are contained within a same nucleic acid molecule. In some instances, the sequence encoding the TFP and the sequence encoding the TCR constant domain are contained within different nucleic acid molecules.

In some instances, the TCR subunit and the antibody domain, the antigen domain or the binding ligand or fragment thereof are operatively linked by a linker sequence. In some instances, the linker sequence comprises (G4S)n, wherein n=1 to 4.

In some instances, the transmembrane domain is a TCR transmembrane domain from CD3 epsilon, CD3 gamma, CD3 delta, TCR alpha or TCR beta. In some instances, the intracellular domain is derived from only CD3 epsilon, only CD3 gamma, only CD3 delta, only TCR alpha or only TCR beta.

In some instances, the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

In some instances, the TCR extracellular domain comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

In some instances, the TCR subunit comprises a transmembrane domain comprising a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

In some instances, the TCR subunit comprises a TCR intracellular domain comprising a stimulatory domain of a protein selected from an intracellular signaling domain of CD3 epsilon, CD3 gamma or CD3 delta, or an amino acid sequence having at least one modification thereto.

In some instances, the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

In some instances, the recombinant nucleic acid further comprises a sequence encoding a costimulatory domain. In some instances, the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

In some instances, the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79a, CD79b, CD89, CD278, CD66d, functional fragments thereof, and amino acid sequences thereof having at least one but not more than 20 modifications thereto. In some instances, the ITAM replaces an ITAM of CD3 gamma, CD3 delta, or CD3 epsilon. In some instances, the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

In some instances, the TFP, the TCR alpha constant domain, the TCR beta constant domain, and any combination thereof is capable of functionally interacting with an endogenous TCR complex and/or at least one endogenous TCR polypeptide. In some instances, (a) the TCR constant domain is a TCR alpha constant domain and the TFP functionally integrates into a TCR complex comprising an endogenous subunit of TCR beta, CD3 epsilon, CD3 gamma, CD3 delta, or a combination thereof, (b) the TCR constant domain is a TCR beta constant domain and the TFP functionally integrates into a TCR complex comprising an endogenous subunit of TCR alpha, CD3 epsilon, CD3 gamma, CD3 delta, or a combination thereof, or (c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

In some instances, the at least one but not more than 20 modifications thereto comprise a modification of an amino acid that mediates cell signaling or a modification of an amino acid that is phosphorylated in response to a ligand binding to the TFP.

In some instances, the human or humanized antibody is an antibody fragment. In some instances, the antibody fragment is a scFv, a single domain antibody domain, a VH domain or a VL domain. In some instances, human or humanized antibody comprising an antigen binding domain is selected from a group consisting of an anti-CD19 binding domain, anti-B-cell maturation antigen (BCMA) binding domain, anti-mesothelin (MSLN) binding domain, anti-CD22 binding domain, anti-BAFF binding domain, anti-PD-1 binding domain, and anti-ROR-1 binding domain.

In some instances, the nucleic acid is selected from the group consisting of a DNA and an RNA. In some instances, the nucleic acid is an mRNA. In some instances, the recombinant nucleic acid comprises a nucleic acid analog, wherein the nucleic acid analog is not in an encoding sequence of the recombinant nucleic acid. In some instances, the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

In some instances, the recombinant nucleic acid further comprises a leader sequence. In some instances, the recombinant nucleic acid further comprises a promoter sequence. In some instances, the recombinant nucleic acid further comprises a sequence encoding a poly(A) tail. In some instances, the recombinant nucleic acid further comprises a 3′UTR sequence. In some instances, the nucleic acid is an isolated nucleic acid or a non-naturally occurring nucleic acid. In some instances, the nucleic acid is an in vitro transcribed nucleic acid.

In some instances, the recombinant nucleic acid further comprises a sequence encoding a TCR alpha transmembrane domain. In some instances, the recombinant nucleic acid further comprises a sequence encoding a TCR beta transmembrane domain. In some instances, the recombinant nucleic acid further comprises a sequence encoding a TCR alpha transmembrane domain and a sequence encoding a TCR beta transmembrane domain.

Further disclosed herein, in some embodiments, are vectors comprising the recombinant nucleic acid disclosed herein. In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In another embodiment, the vector comprising the nucleic acid encoding the desired TFP of the present disclosure is an adenoviral vector (A5/35). In another embodiment, the expression of nucleic acids encoding TFPs can be accomplished using of transposons such as sleeping beauty, crisper, CAS9, and zinc finger nucleases. See below June et al. 2009 Nature Reviews Immunology 9.10: 704-716, is incorporated herein by reference.

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties). In another embodiment, the present disclosure provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, Molecular Cloning: A Laboratory Manual, volumes 1-4, Cold Spring Harbor Press, NY). A preferred method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and western blots) or by assays described herein to identify agents falling within the scope of the present disclosure.

The present disclosure further provides a vector comprising a TFP encoding nucleic acid molecule. In one aspect, a TFP vector can be directly transduced into a cell, e.g., a T cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the TFP construct in mammalian T cells. In one aspect, the mammalian T cell is a human T cell.

### Modified T Cells

Disclosed herein, in some embodiments, are modified T cells comprising the recombinant nucleic acid disclosed herein, or the vectors disclosed herein; wherein the modified T cell comprises a functional disruption of an endogenous TCR. Also disclosed herein, in some embodiments, are modified T cells comprising the sequence encoding the TFP of the nucleic acid disclosed herein or a TFP encoded by the sequence of the nucleic acid disclosed herein, wherein the modified T cell comprises a functional disruption of an endogenous TCR. Further disclosed herein, in some embodiments, are modified allogenic T cells comprising the sequence encoding the TFP disclosed herein or a TFP encoded by the sequence of the nucleic acid disclosed herein.

In some instances, the T cell further comprises a heterologous sequence encoding a TCR constant domain, wherein the TCR constant domain is a TCR alpha constant domain, a TCR beta constant domain or a TCR alpha constant domain and a TCR beta constant domain. In some instances, the endogenous TCR that is functionally disrupted is an endogenous TCR alpha chain, an endogenous TCR beta chain, or an endogenous TCR alpha chain and an endogenous TCR beta chain. In some instances, the endogenous TCR that is functionally disrupted has reduced binding to MHC-peptide complex compared to that of an unmodified control T cell. In some instances, the functional disruption is a disruption of a gene encoding the endogenous TCR. In some instances, the disruption of a gene encoding the endogenous TCR is a removal of a sequence of the gene encoding the endogenous TCR from the genome of a T cell. In some instances, the T cell is a human T cell. In some instances, the T cell is a CD8+ or CD4+ T cell. In some instances, the T cell is an allogenic T cell. In some instances, the modified T cells further comprise a nucleic acid encoding an inhibitory molecule that comprises a first polypeptide comprising at least a portion of an inhibitory molecule, associated with a second polypeptide comprising a positive signal from an intracellular signaling domain. In some instances, the inhibitory molecule comprises the first polypeptide comprising at least a portion of PD1 and the second polypeptide comprising a costimulatory domain and primary signaling domain.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T cell lines available in the art, may be used. In certain aspects of the present disclosure, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the present disclosure, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe® 2991 cell processor, the Baxter OncologyCytoMate, or the Haemonetics® Cell Saver® 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed, and the cells directly resuspended in culture media.

In one aspect, T cells are isolated from peripheral blood lymphocytes by lysing the red blood cells and depleting the monocytes, for example, by centrifugation through a PERCOLL® gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this present disclosure. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

In one embodiment, a T cell population can be selected that expresses one or more of IFN-γ TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/mL is used. In one aspect, a concentration of 1 billion cells/mL is used. In a further aspect, greater than 100 million cells/mL is used. In a further aspect, a concentration of cells of 10, 15, 20, 25, 30, 35, 40, 45, or 50 million cells/mL is used. In yet one aspect, a concentration of cells from 75, 80, 85, 90, 95, or 100 million cells/mL is used. In further aspects, concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/mL. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. In certain aspects, cryopreserved cells are thawed and washed as described herein and allowed to rest for one hour at room temperature prior to activation using the methods of the present disclosure.

Also contemplated in the context of the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents such as cyclosporin, azathioprine, methotrexate, and mycophenolate, antibodies, or other immunoablative agents such as alemtuzumab, anti-CD3 antibodies, cytoxan, fludarabine, cyclosporin, tacrolimus, rapamycin, mycophenolic acid, steroids, romidepsin, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631.

Generally, the T cells of the present disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-CD19 anti-BCMA, anti-CD22, anti-ROR1, anti-PD-1, or anti-BAFF TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-CD19 anti-BCMA, anti-CD22, anti-ROR1, anti-PD-1, or anti-BAFF TFP are described in further detail below

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4+ and CD8+ T cells) expressing the TFPs are expanded in vitro for more than 10 days followed by lysis and SDS-PAGE under reducing conditions. TFPs are detected by western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-lalpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0, and transduced with TFP on day 1 using a bicistronic lentiviral vector expressing TFP along with eGFP using a 2A ribosomal skipping sequence. Cultures are re-stimulated with either CD19+K562 cells (K562-CD19), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human CD19-specific TFP+ T cells to treat a primary human pre-B ALL in immunodeficient mice can be used (see, e.g., Milone et al., Molecular Therapy 17(8): 1453-1464 (2009)). Very briefly, after establishment of ALL, mice are randomized as to treatment groups. Different numbers of engineered T cells are coinjected at a 1:1 ratio into NOD/SCID/γ−/− mice bearing B-ALL. The number of copies of each vector in spleen DNA from mice is evaluated at various times following T cell injection. Animals are assessed for leukemia at weekly intervals. Peripheral blood CD19+B-ALL blast cell counts are measured in mice that are injected with alphaCD19-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with leukemic cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for leukemia at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing leukemia in mice injected on day 21 with TFP T cells, an equivalent number of mock-transduced T cells, or no T cells. Mice from each group are randomly bled for determination of peripheral blood CD19+ ALL blast counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with K562 cells expressing CD19 (K19) or CD32 and CD137 (KT32-BBL) for a final T cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant CD19 protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur™ flow cytometer (BD Biosciences), and data are analyzed according to the manufacturer's instructions.

Cytotoxicity can be assessed by a standard ⁵¹Cr-release assay (see, e.g., Milone et al., Molecular Therapy 17(8): 1453-1464 (2009)). Target cells (K562 lines and primary pro-B-ALL cells) are loaded with ⁵¹Cr (as NaCrO₄, New England Nuclear) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of Triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). NOD/SCID/γc−/− (NSG) mice are injected IV with Nalm-6 cells (ATCC® CRL-3273™) followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in Nalm-6 xenograft model can be measured as the following: NSG mice are injected with Nalm-6 transduced to stably express firefly luciferase, followed by a single tail-vein injection of T cells electroporated with CD19 TFP 7 days later. Animals are imaged at various time points post injection. For example, photon-density heat maps of firefly luciferase positive leukemia in representative mice at day 5 (2 days before treatment) and day 8 (24 hours post TFP+ PBLs) can be generated.

Other assays, including those described in the Example section herein as well as those that are known in the art can also be used to evaluate the anti-CD19, anti-BCMA, anti-CD22, anti-ROR1, anti-PD-1, or anti-BAFF TFP constructs disclosed herein.

### Pharmaceutical Compositions

Disclosed herein, in some embodiments, are pharmaceutical compositions comprising: (a) the modified T cells of the disclosure; and (b) a pharmaceutically acceptable carrier. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are in one aspect formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 105 to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions of the present disclosure are administered to a patient by intradermal or subcutaneous injection. In one aspect, the T cell compositions of the present disclosure are administered by i.v. injection. The compositions of T cells may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a modified T-T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded modified T cells of the present disclosure. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the TFP is introduced into T cells, e.g., using in vitro transcription, and the subject (e.g., human) receives an initial administration of TFP T cells of the present disclosure, and one or more subsequent administrations of the TFP T cells of the present disclosure, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the TFP T cells of the present disclosure are administered to the subject (e.g., human) per week, e.g., 2, 3, or 4 administrations of the TFP T cells of the present disclosure are administered per week. In one embodiment, the subject (e.g., human subject) receives more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. In another embodiment, the subject (e.g., human subject) receives more than one cycle of TFP T cells, and the time between each cycle is less than 10, 9, 8, 7, 6, 5, 4, or 3 days. In one embodiment, the TFP T cells are administered every other day for 3 administrations per week. In one embodiment, the TFP T cells of the present disclosure are administered for at least two, three, four, five, six, seven, eight or more weeks.

In one aspect, CD19 TFP T cells are generated using lentiviral viral vectors, such as lentivirus. TFP-T cells generated that way will have stable TFP expression.

In one aspect, TFP T cells transiently express TFP vectors for 4, 5, 6, 7, 8, 9, 10, 11, 12, 13, 14, 15 days after transduction. Transient expression of TFPs can be effected by RNA TFP vector delivery. In one aspect, the TFP RNA is transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Methods of Producing Modified T Cells

Disclosed herein, in some embodiments, are methods of producing the modified T cell of the disclosure, the method comprising (a) disrupting an endogenous TCR gene encoding a TCR alpha chain, a TCR beta chain, a TCR gamma chain, a TCR delta chain, or any combination thereof, thereby producing a T cell containing a functional disruption of an endogenous TCR gene; and (b) transducing the T cell containing a functional disruption of an endogenous TCR gene with the recombinant nucleic acid of the disclosure, or the vectors disclosed herein. In some instances, disrupting comprises transducing the T cell with a nuclease protein or a nucleic acid sequence encoding a nuclease protein that targets the endogenous gene encoding a TCR alpha chain, a TCR beta chain, or a TCR alpha chain and a TCR beta chain.

Further disclosed herein, in some embodiments, are methods of producing the modified T cell of the disclosure, the method comprising transducing a T cell containing a functional disruption of an endogenous TCR gene with the recombinant nucleic acid disclosed herein, or the vectors disclosed herein. In some instances, the T cell containing a functional disruption of an endogenous TCR gene is a T cell containing a functional disruption of an endogenous TCR gene encoding a TCR alpha chain, a TCR beta chain, or a TCR alpha chain and a TCR beta chain.

In some instances, the T cell is a human T cell. In some instances, the T cell containing a functional disruption of an endogenous TCR gene has reduced binding to MHC-peptide complex compared to that of an unmodified control T cell.

In some instances, the nuclease is a meganuclease, a zinc-finger nuclease (ZFN), a transcription activator-like effector nuclease (TALEN), a CRISPR/Cas nuclease, CRISPR/Cas nickase, or a megaTAL nuclease. In some instances, the sequence comprised by the recombinant nucleic acid or the vector is inserted into the endogenous TCR subunit gene at the cleavage site, and wherein the insertion of the sequence into the endogenous TCR subunit gene functionally disrupts the endogenous TCR subunit. In some instances, the nuclease is a meganuclease. In some instances, the meganuclease comprises a first subunit and a second subunit, wherein the first subunit binds to a first recognition half-site of the recognition sequence, and wherein the second subunit binds to a second recognition half-site of the recognition sequence. In some instances, the meganuclease is a single-chain meganuclease comprising a linker, wherein the linker covalently joins the first subunit and the second subunit.

### Gene Editing Technologies

In some embodiments, the modified T cells disclosed herein are engineered using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. In this way, a chimeric construct may be engineered to combine desirable characteristics of each subunit, such as conformation or signaling capabilities. See also Sander & Joung, Nat. Biotech. (2014) v32, 347-55; and June et al., 2009 Nature Reviews Immunol. 9.10: 704-716, each incorporated herein by reference. In some embodiments, one or more of the extracellular domain, the transmembrane domain, or the cytoplasmic domain of a TFP subunit are engineered to have aspects of more than one natural TCR subunit domain (i.e., are chimeric).

Recent developments of technologies to permanently alter the human genome and to introduce site-specific genome modifications in disease relevant genes lay the foundation for therapeutic applications. These technologies are now commonly known as “genome editing.

In some embodiments, gene editing techniques are employed to distrupt an endogenous TCR gene. In some embodiments, mentioned endogenous TCR gene encodes a TCR alpha chain, a TCR beta chain, or a TCR alpha chain and a TCR beta chain. In some embodiments, gene editing techniques pave the way for multiplex genomic editing, which allows simultaneous disruption of multiple genomic loci in endogenous TCR gene. In some embodiments, multiplex genomic editing tecniques are applied to generate gene-disrupted T cells that are deficient in the expression of endogenous TCR, and/or human leukocyte antigens (HLAs), and/or programmed cell death protein 1 (PD1), and/or other genes.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or—when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a pre-determined DNA sequence −18 basepairs in length. By fusing this engineered protein domain to the Fokl nuclease, it is possible to target DNA breaks with genome-level specificity. ZFNs have been used extensively to target gene addition, removal, and substitution in a wide range of eukaryotic organisms (reviewed in Durai et al. (2005), Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fokl nuclease domain (reviewed in Mak et al. (2013), Curr Opin Struct Biol. 23:93-9). In this case, however, the DNA binding domain comprises a tandem array of TAL-effector domains, each of which specifically recognizes a single DNA basepair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-TevI homing endonuclease. Unlike Fokl, I-TevI does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or a RNA duplex comprising a 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally-occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy. 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, must be efficiently delivered to the cells of interest. Delivery methods such as physical, chemical, and viral methods are also know in the art (Mali (2013). Indian J. Hum. Genet. 19: 3-8.). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

### Methods of Treatment

Disclosed herein, in some embodiments, are methods of treating cancer in a subject in need thereof, the method comprising administering to the subject a therapeutically effective amount of the pharmaceutical compositions disclosed herein. Further disclosed herein, in some embodiments, are methods of treating cancer in a subject in need thereof, the method comprising administering to the subject a pharmaceutical composition comprising (a) a modified T cell produced according to the methods disclosed herein; and (b) a pharmaceutically acceptable carrier.

In some instances, the modified T cell is an allogeneic T cell. In some instances, less cytokines are released in the subject compared a subject administered an effective amount of an unmodified control T cell. In some instances, less cytokines are released in the subject compared a subject administered an effective amount of a modified T cell comprising the recombinant nucleic acid disclosed herein, or the vector disclosed herein.

In some instances, the method comprises administering the pharmaceutical composition in combination with an agent that increases the efficacy of the pharmaceutical composition. In some instances, the method comprises administering the pharmaceutical composition in combination with an agent that ameliorates one or more side effects associated with the pharmaceutical composition.

In some instances, the cancer is a solid cancer, a lymphoma or a leukemia. In some instances, the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

The present disclosure includes a type of cellular therapy where T cells are genetically modified to express a TFP and a TCR alpha and/or beta constant domain and the modified T cell is infused to a recipient in need thereof. The infused cell is able to kill tumor cells in the recipient. Unlike antibody therapies, modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The present disclosure also includes a type of cellular therapy where T cells are modified, e.g., by in vitro transcribed RNA, to transiently express a TFP and a TCR alpha and/or beta constant domain and the modified T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the modified T cells may be an active or a passive immune response, or alternatively may be due to a direct vs indirect immune response.

In one aspect, the human modified T cells of the disclosure may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a TFP and a TCR alpha and/or beta constant domain to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector disclosed herein. The modified T cell can be administered to a mammalian recipient to provide a therapeutic benefit. The mammalian recipient may be a human and the modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised.

The modified T cells of the present disclosure may be administered either alone, or as a pharmaceutical composition in combination with diluents and/or with other components such as IL-2 or other cytokines or cell populations.

### Combination Therapies

A modified T cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the “at least one additional therapeutic agent” includes a modified T cell. Also provided are T cells that express multiple TFPs, which bind to the same or different target antigens, or same or different epitopes on the same target antigen. Also provided are populations of T cells in which a first subset of T cells express a first TFP and a TCR alpha and/or beta constant domain and a second subset of T cells express a second TFP and a TCR alpha and/or beta constant domain.

A modified T cell described herein and the at least one additional therapeutic agent can be administered simultaneously, in the same or in separate compositions, or sequentially. For sequential administration, the modified T cell described herein can be administered first, and the additional agent can be administered second, or the order of administration can be reversed.

In further aspects, a modified T cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. peptide vaccine, such as that described in Izumoto et al. 2008 J Neurosurg 108:963-971.

In one embodiment, the subject can be administered an agent which reduces or ameliorates a side effect associated with the administration of a modified T cell. Side effects associated with the administration of a modified T cell include but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods disclosed herein can comprise administering a modified T cell described herein to a subject and further administering an agent to manage elevated levels of a soluble factor resulting from treatment with a modified T cell. In one embodiment, the soluble factor elevated in the subject is one or more of IFN-γ, TNFα, IL-2 and IL-6. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

In one embodiment, the subject can be administered an agent which enhances the activity of a modified T cell. For example, in one embodiment, the agent can be an agent which inhibits an inhibitory molecule. Inhibitory molecules, e.g., Programmed Death 1 (PD1), can, in some embodiments, decrease the ability of a modified T cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a modified T cell performance. In embodiments, an inhibitory nucleic acid, e.g., an inhibitory nucleic acid, e.g., a dsRNA, e.g., an siRNA or shRNA, can be used to inhibit expression of an inhibitory molecule in the TFP-expressing cell. In an embodiment the inhibitor is a shRNA. In an embodiment, the inhibitory molecule is inhibited within a modified T cell. In these embodiments, a dsRNA molecule that inhibits expression of the inhibitory molecule is linked to the nucleic acid that encodes a component, e.g., all of the components, of the TFP. In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3. In an embodiment, the agent is an antibody or antibody fragment that binds to LAG3.

In some embodiments, the agent which enhances the activity of a modified T cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the TFP. In another embodiment, the fusion protein is expressed by a cell, e.g., a T cell that does not express an anti-CD19 TFP.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Background for Examples 1-5

T cell receptors (TCRs) recognize foreign antigens which have been processed as small peptides and bound to major histocompatibility complex (MHC) molecules at the surface of antigen presenting cells (APCs). The T cell receptor (TCR) complex is formed by a grouping of dimers, including: T cell receptor alpha and beta subunits (TCRα/β) or gamma and delta subunits (TCRγδ); and CD3 dimers CD3γ/ε, CD3δ/ε, and CD3ζ/ζ. The T cell receptor alpha constant (TRAC) and T cell receptor beta constant (TRBC) genes encode for the constant C-terminal region of TCRα and TCRβ, respectively.

Disruption of the TCR constant region(s) blocks the translocation of TCRα or TCRβ to the cell surface, thus inhibiting assembly of the TCR receptor complex. Impairing the translocation of TCRα or TCRβ is enough to inhibit the assembling of entire TCR receptor. Inactivation of the TCR complex may therefore be done by targeting the TRAC or TRBC genes with a gene editing method using clustered regularly interspaced short palindromic repeat (CRISPR) method, transcription activator-like effector nucleases (TALENs), zinc finger nucleases or meganucleases. However, TFP T cells based on CD3ε or CD3γ or CD3δ fusion proteins require surface expression of TCR α/β to incorporate into a functional TCR complex.

Activation of the TCR complex on the surface of donor T cells by receiver antigens (i.e., recognition of antigens presented by the major histocompatibility complex (MHC) on antigen presenting cells) can trigger unwanted effects such as graft-versus-host disease (GvHD) and cytokine release syndrome (CRS). Thus, the following Examples describe methods of introducing a transgene in TCR knock-out cells encoding for a truncated version of TCRα or TCRβ, and the fusion protein itself separated by a self-cleavage signal (e.g., T2A). In one embodiment, the truncated version of TCRα or TCRβ includes the transmembrane domain and the connecting peptide domain (CP) of TCRα or TCRβ. In another embodiment, the TFP's antigen binding domain is fused at the N-terminal end of the truncated or full length TCRα and/or TCRβ.

### Example 1. crRNA (CRISPR RNA) Design

crRNAs to inactivate TRA were designed with “Dunne 2017” algorithm accessible on DeskGen™ CRISPR library website (www.deskgen.com). Any crRNAs binding the TRA locus are able to efficiently generate double strand breaks in the TRA gene. To minimize off-target activity of the CRISPR endonuclease, crRNAs used have an off-target score of >9000, comprising at least 3 mismatches with the closest homolog sequence in the Genome Reference Consortium Human genome build 38 (GRCh38/hg38) genome. In a preferred embodiment, one mismatch is located in the 8 bp upstream to the protospacer adjacent motif (PAM). Tables 1-2 show exemplary crRNA sequences selected to inactivate the TRA gene (Table 1) and predicted off target activity (Table 2).

TABLE 1
crRNAs selected to inactivate TRA gene:

| | | | | | Off |
|---|---|---|---|---|---|
| | | | | | target |
| | | | | | score |
| ID | crRNA | PAM | Target | Genomic Location | (%) |
| TRAC1- | TCTCTCAGCTGGTACACGGC | AGG | TRAC1 | chr14: 22547526- | 94 |
| 4894 | | | | 22547545 | |
| TRAC2- | CTCGACCAGCTTGACATCAC | AGG | TRAC2 | chr14: 22549647- | 98 |
| 4598 | | | | 22549666 | |
| TRAC3- | GATTAAACCCGGCCACTTTC | AGG | TRAC3 | chr14: 22550612- | 98 |
| 2998 | | | | 22550631 | |

TABLE 2
Predicted off-target sites; mismatches between on and off-target are
indicated in bold

| crRNA | Off-Target | PAM | Mismatch | Exon | Genomic Location |
|---|---|---|---|---|---|
| TRAC1-4894 | TCCCTCAGCTGGTACAAGGA | TGG | 3, 17, 20 | Yes | chr1: 186070730- |
| | | | | | 186070753 |
| | TCTGTCAACTGGTACATGGC | AAG | 4, 8 ,17 | No | chrX: 83244396- |
| | | | | | 83244419 |
| | TCTCATAGCTGGTACATGGC | GGG | 5, 6, 17 | No | chr15: 100865579- |
| | | | | | 100865602 |
| | TTTCTCAGCTGGTACATGGA | GGG | 2, 17, 20 | No | chr1: 247923608- |
| | | | | | 247923631 |
| | GCACTCAGCTGGTACCCGGC | AAG | 1, 3, 16 | No | chr16: 8713603- |
| | | | | | 8713626 |
| | TCACTCAGCTGGTACATGGG | CAG | 3, 17, 20 | No | chr4: 130310607- |
| | | | | | 130310630 |
| | TCTCCCAGCTGGGACACGGT | GAG | 5, 13, 20 | No | chr1: 55167399- |
| | | | | | 55167422 |
| | TCAATCAGCTGGTGCACGGC | TGG | 3, 4, 14 | No | chr1: 236924538- |
| | | | | | 236924561 |
| | TCTCACAGCTGATATACGGC | TGG | 5, 12, 15 | No | chr12: 49641344- |
| | | | | | 49641367 |
| TRAC2-4598 | CTCCACCACCTTGACCTCAC | CGG | 4, 9, 16 | Yes | chr10: 102422239- |
| | | | | | 102422262 |
| | CTCAACCAGAATGACATCAC | CAG | 4, 10, 11 | No | chr2: 55715822- |
| | | | | | 55715845 |
| | CTAGACCAGCTTGACCTCCC | CAG | 3, 16, 19 | No | chr4: 89585943- |
| | | | | | 89585966 |
| | CTAGACCAGCTTGGCAACAC | AGG | 3, 14, 17 | No | chr5: 82123725- |
| | | | | | 82123748 |
| TRAC3-2998 | GAATAAAACCGGCCACTTTG | GGG | 3, 8, 20 | No | chr5: 128101267- |
| | | | | | 128101290 |
| | GATTATACCTGGCCACATTC | AAG | 6, 10, 17 | No | chr2: 145719958- |
| | | | | | 145719981 |

crRNAs to inactivate TRB were designed with Dunne 2017 algorithm as described above. As the constant region of TCRβ is encoded by two genes, TRBC1 and TRBC2, crRNAs are directed against sequences identical in both TRBC1 and TRBC2. Consequently, the off-target score generated by DeskGen™ is lower than 94%. However, aside from targeting TRBC1 and TRBC2, other homolog sequences between crRNAs and the GRCh38/hg38 genome carry at least 3 mismatches. In a preferred embodiment, one of those mismatches is localized in the 8 bp upstream to the Protospacer adjacent motif (PAM). Tables 3-4 show exemplary crRNA sequences selected to inactivate the TRB gene (Table 3) and predicted off target activity (Table 4).

TABLE 3
crRNAs selected to inactivate TRB gene

| | | | | | Off- |
|---|---|---|---|---|---|
| | | | | | target |
| | | | | | score |
| ID | crRNA | PAM | Target | Location | (%) |
| TRBC- | ACACTGGTGTGCCTGGC | AGG | TRBC1 | chr7: 142801121-142801140 | 45 |
| 44345 | CAC | | TRBC2 | chr7_KI270803v1_alt: 814747- | |
| | | | | 814766 | |
| TRBC- | AGGGCGGGCTGCTCCTT | GGG | TRBC1 | chr7: 142791879-142791898 | 47 |
| 45447 | GAG | | TRBC2 | chr7: 142801226-142801245 | |
| a | | | | | |
| TRBC- | CTGCCTGAGCAGCCGCC | GGG | TRBC1 | chr7: 142791914-142791933 | 46 |
| 45246 | TGA | | TRBC2 | chr7: 142801261-142801280 | |
| TRBC- | GCGGGGGTTCTGCCAGA | TGG | TRBC1 | chr7: 142791946-142791965 | 47 |
| 45447 | AGG | | TRBC2 | chr7: 142801293-142801312 | |
| b | | | | | |

TABLE 4
Predicted off-targets, mismatches between on and off-target are indicted in bold

| crRNA | Off-Target | PAM | Mismatch | Exon | Locus |
|---|---|---|---|---|---|
| TRBC-44345 | ACTCTGGGCTGCCTGGCCAC | GGG | 3, 8, 9 | Yes | chr14: 105601630- |
| | | | | | 105601653 |
| | ACTCTGTTGTGCCTGGACAC | CGG | 3, 7, 17 | Yes | chr20: 62963310- |
| | | | | | 62963333 |
| | TCACAGGTGAGCCTGGCCAC | AGG | 1, 5, 10 | No | chr14: 98950719- |
| | | | | | 98950742 |
| | GCACGGGTGGGCCTGGCCAC | TGG | 1, 5, 10 | No | chr12: 108839394- |
| | | | | | 108839417 |
| | GCAGGGGTGTGCCTGGCCAC | TGG | 1, 4, 5 | No | chr16: 3010877- |
| | | | | | 3010900 |
| | ATCCTGCTGTGCCTGGCCAC | AGG | 2, 3, 7 | No | chr6: 37655368- |
| | | | | | 37655391 |
| | TCTCTGGTGTGCCTGGCCAA | GAG | 1, 3, 20 | No | chrX: 138046658- |
| | | | | | 138046681 |
| | ACACATGTGGGCCTGGCCAC | GGG | 5, 6, 10 | No | chr16: 2438272- |
| | | | | | 2438295 |
| | AGCCTGGTGTGTCTGGCCAC | TGG | 2, 3, 12 | No | chr2: 162055950- |
| | | | | | 162055973 |
| | CCTCTGGTGTGCCTGGCCCC | AGG | 1, 3, 19 | No | chr2: 239228091- |
| | | | | | 239228114 |
| | CCACTTGTGTGCATGGCCAC | TAG | 1, 6, 13 | No | chr1: 101657244- |
| | | | | | 101657267 |
| | ATAATGGTGTGCCTGGCAAC | TAG | 2, 4, 18 | No | chr1: 230924183- |
| | | | | | 230924206 |
| | ACACTGGCCTGCCTGGGCAC | TAG | 8, 9, 17 | No | chr1: 155926881- |
| | | | | | 155926904 |
| TRBC-45447a | AGCGCGGGCTCCTCCTTGAC | GGG | 3, 11, 20 | Yes | chr8: 143598506- |
| | | | | | 143598529 |
| | AGGGCCTGCTGCTCCTTCAG | CAG | 6, 7, 18 | Yes | chr3: 45030894- |
| | | | | | 45030917 |
| | AGGGCTGACAGCTCCTTGAG | TGG | 6, 8, 10 | No | chr20: 683139-683162 |
| | GGGGTGGGCTGCTCCTGGAG | CAG | 1, 5, 17 | No | chr20: 63440195- |
| | | | | | 63440218 |
| | AGAGCGGCCTGCTCCTCGAG | GGG | 3, 8, 17 | No | chr17: 50124057- |
| | | | | | 50124080 |
| | GGGGTGGGCTGCACCTTGAG | GGG | 1, 5, 13 | No | chr12: 3189255- |
| | | | | | 3189278 |
| | AAGGCAGGCTCCTCCTTGAG | AGG | 2, 6, 11 | No | chr5: 176733401- |
| | | | | | 176733424 |
| | AGGAAGGGCTGCTCTTTGAG | GAG | 4, 5, 15 | No | chr10: 100783415- |
| | | | | | 100783438 |
| | AGGCTGGGCTGCTCTTTGAG | CAG | 4, 5, 15 | No | chr1: 226617392- |
| | | | | | 226617415 |
| | AGTGCCGGCTGCTCCTGGAG | TGG | 3, 6, 17 | No | chr15: 74624787- |
| | | | | | 74624810 |
| | AGGGTGGGGTGCTCCTCGAG | GGG | 5, 9, 17 | No | chr7: 99165433- |
| | | | | | 99165456 |
| | TGGGCTGGCTGCACCTTGAG | TAG | 1, 6, 13 | No | chr12: 92396203- |
| | | | | | 92396226 |
| | TGGGCGGGCTGTTCCTTGGG | GAG | 1, 12, 19 | No | chr5: 179287136- |
| | | | | | 179287159 |
| TRBC-45246 | CTTCCTGAGCAGCCGTCTGC | AGG | 3, 16, 20 | Yes | chr5: 177525051- |
| | | | | | 177525074 |
| | CTGCCTGAGCAGCTGCCACA | AGG | 14, 18, 19 | Yes | chr21: 42085445- |
| | | | | | 42085468 |
| | CAGCGTTAGCAGCCGCCTGA | GGG | 2, 5, 7 | No | chr6: 24719514- |
| | | | | | 24719537 |
| | CACCCAGAGCAGCCGCCTGA | CAG | 2, 3, 6 | No | chr8: 58226030- |
| | | | | | 58226053 |
| | CTGCCTGGGAAGCCGCCTGC | CAG | 8, 10, 20 | No | chr1: 41873106- |
| | | | | | 41873129 |
| | CTGCCTCCTCAGCCGCCTGA | GGG | 7, 8, 9 | No | chr15: 89663036- |
| | | | | | 89663059 |
| | CTGTCTGACCAGCCGCCTGC | CGG | 4, 9, 20 | No | chr1: 9401937-9401960 |
| | CAGCCTGAGCTGCCGCCTGC | GGG | 2, 11, 20 | No | chr17: 36923765- |
| | | | | | 36923788 |
| | CAACCTGAGCAGCCTCCTGA | GAG | 2, 3, 15 | No | chr8: 127075998- |
| | | | | | 127076021 |
| | CTCCCTGATCAGCCGCATGA | GGG | 3, 9, 17 | No | chr20: 63598726- |
| | | | | | 63598749 |
| | CGGCCGGAGCAGCCGCCTCA | GGG | 2, 6, 19 | No | chr1: 204685196- |
| | | | | | 204685219 |
| | CTGCCTCAACATCCGCCTGA | AAG | 7, 9, 12 | No | chrX: 58268037- |
| | | | | | 58268060 |
| TRBC-44547b | GTTGGGATTCTGCCAGAAGG | CAG | 2, 3, 7 | No | chr17: 52505137- |
| | | | | | 52505160 |
| | GAGGGGGGCCTGCCAGAAGG | AGG | 2, 8, 9 | No | chr8: 1547518-1547541 |
| | GCGGAAGATCTGCCAGAAGG | GGG | 5, 6, 8 | No | chr16: 1946717- |
| | | | | | 1946740 |
| | GGTGGGGTTCTGCCAGGAGG | AGG | 2, 3, 17 | No | chr9: 135224974- |
| | | | | | 135224997 |
| | GCGGGGGATGTGCCAGGAGG | AGG | 8, 10, 17 | No | chr11: 62414927- |
| | | | | | 62414950 |
| | GAGGGGATTCTGCCAGCAGG | CGG | 2, 7, 17 | No | chr5: 133192714- |
| | | | | | 133192737 |
| | GAGGGGGTCCTGCCAGCAGG | GAG | 2, 9, 17 | No | chr6: 13415078- |
| | | | | | 13415101 |
| | GAGGGTGTTCTGCCAGCAGG | CAG | 2, 6, 17 | No | chr8: 23039425- |
| | | | | | 23039448 |
| | GCAGGGGTTCAGCCAGGAGG | CAG | 3, 11, 17 | No | chr11: 60938213- |
| | | | | | 60938236 |
| | GAGGGGGTTCAGACAGAAGG | CAG | 2, 11, 13 | No | chr18: 13654430- |
| | | | | | 13654453 |
| | GCAGGGGTTCTCCCAGTAGG | CAG | 3, 12, 17 | No | chr3:18516713- |
| | | | | | 18516736 |
| | GTGGGGGTTCTGCCAGCAGC | TGG | 2, 17, 20 | No | chr17: 68030673- |
| | | | | | 68030696 |

### Example 2: Editing of Endogenous TCRα or β in Jurkat Cells

Inactivation of the TRA or TRB genes in Jurkat cells was done by electroporation of SpCas9 ribonucleoproteins (RNPs) directed against TRA or TRB genes. Cells were maintained at 0.2×10⁶ cells per mL in RPMI 1640 medium supplemented with 10% Fetal Bovine Serum (FBS) and 300 mg/L L-Glutamine until electroporation. SpCas9 ribonucleoproteins targeting TRA or TRB genes were prepared by annealing crRNA targeting either TRAC (TRAC2-4598) or TRBC (TRBC-44345) with tracrRNA at a molecular ratio of 1:1. Annealed duplexes were mixed with SpCas9 protein at a molecular ratio of 1.5:1. 0.61 μM of RNPs were mixed with 2.5×10⁶ T cells and electroporated according to the manufacturer's protocol for the Neon Transfection System (ThermoFisher). Electroporation was set at 1600V, 10 ms, 3 pulses. After pulse the cells were immediately transferred to warm medium and incubated at 37° C. for three days.

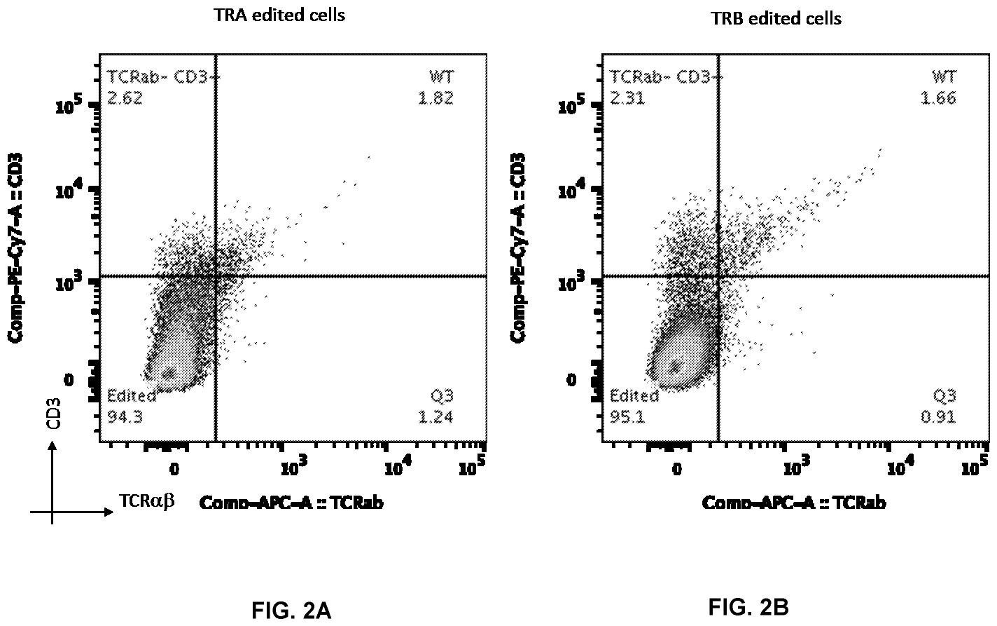

Editing efficacy was assessed by observing loss of surface expression of TCRαβ and CD3ε via flow cytometry. Results are shown in FIG. 2 for TRA edited cells (left panel) and TRB edited cells (right panel). Edited Jurkat cells were purified via Magnetic-Activated Cell Sorting (MACS, Miltenyi Biotec) cell separation system. Edited Jurkat cells were negatively selected against anti-TCRαβ IP27 (eBioscience #17-9986-42) antibody and anti-CD3ε SK7 antibody (eBioscience #25-0036-42). Cells expressing TCRαβ or CD3ε at their surface were immobilized to MACS MS (Cat. #130-041-301) or LS (Cat. #130-041-306) columns, while edited Jurkat cells, negative for both TCRαβ and CD3ε, were collected in the column flow through and maintained in culture at 0.4×10⁶ cells/mL in the medium specified above.

### Example 3. Editing of Human T Cells

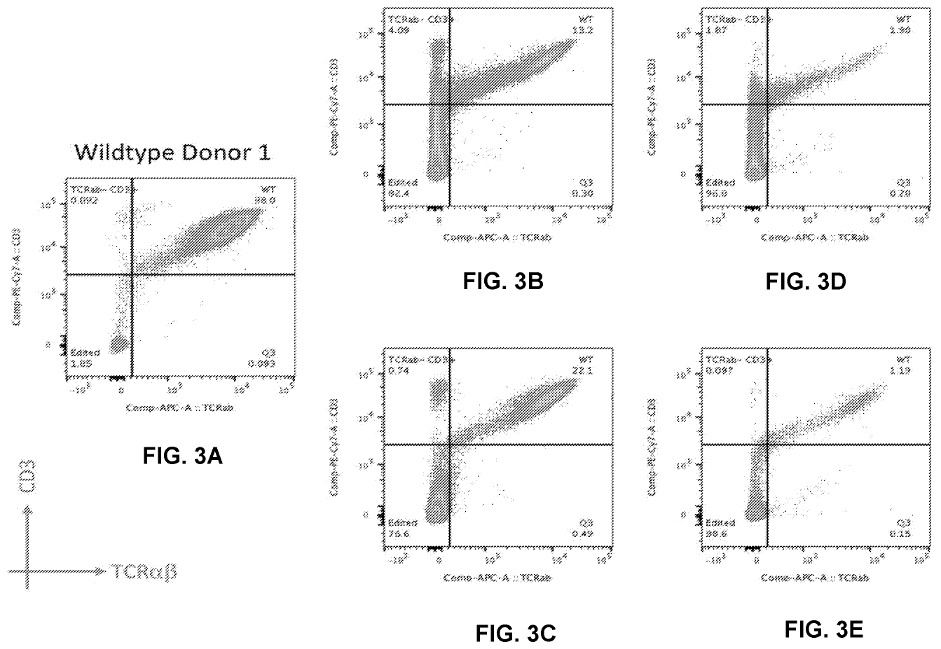

TRA or TRB genes are then inactivated in primary T cells from a human donor. Two to four days prior to electroporation, T cells were activated with Dynabeads® human T cell activator beads specific to CD3/CD28 (Gibco #11132D) at a ratio of 1:1 in CTS optimizer media (Gibco #A1022101) complemented with 10% of human serum (hAB, Valley Biomedical HP1022) and 300 U·ml⁻¹ ITL2 (Petrotech #200-02). SpCas9 ribonucleoproteins (RNPs) targeting TRA or TRB genes were prepared by annealed crRNA targeting either TRAC (TRAC2-4598) or TRBC (TRBC-44345) with tracrRNA at a molecular ratio of 1:1. Annealed duplexes were mixed with SpCas9 protein at a molecular ratio of 1.5:1. 0.61 μM of RNPs were mixed with 2.5×10⁶ T cells and electroporated following the manufacturer's protocol for the Neon Transfection System, electroporation was set at 1600V, 10 ms, 3 pulses. Cells were immediately transferred to warm medium (CTS Optimizer (Gibco #A1048501) with 10% hAB (Valley Biomedical #11P1022), 300 u·ml⁻¹ IL2(Petrotech #200-02), 25 ng·mL⁻¹IL7 (R & D System #207-IL-010) and incubated at 37° C. to allow expansion of edited T cells with an approximate doubling time of 3 to 5 days. Editing efficacy was assessed by measuring loss of surface expression of TCRαβ and CD3ε via flow cytometry. Edited T cells were purified using Magnetic-Activated Cell Sorting (MACS®, Miltenyi Biotec) according to the manufacturer. cell separation system and were negatively selected against anti-TCRαβ IP27 antibody (eBioscience #14-9986-82) and anti-CD3ε SK7 antibodies (eBioscience #16-0036-81). Cells expressing TCRαβ or CD3ε at their surface were immobilized on MACS MS (Cat. #130-041-301) or LS (Cat#130-041-306) columns, while edited T cells, both negative for TCRαβ and CD3ε, were collected in the column flow-through and maintained in culture at 10⁶ cells/mL in the medium specified above. Results are shown in FIG. 3.

### Example 4: Allogenicity of TCR Negative T Cells

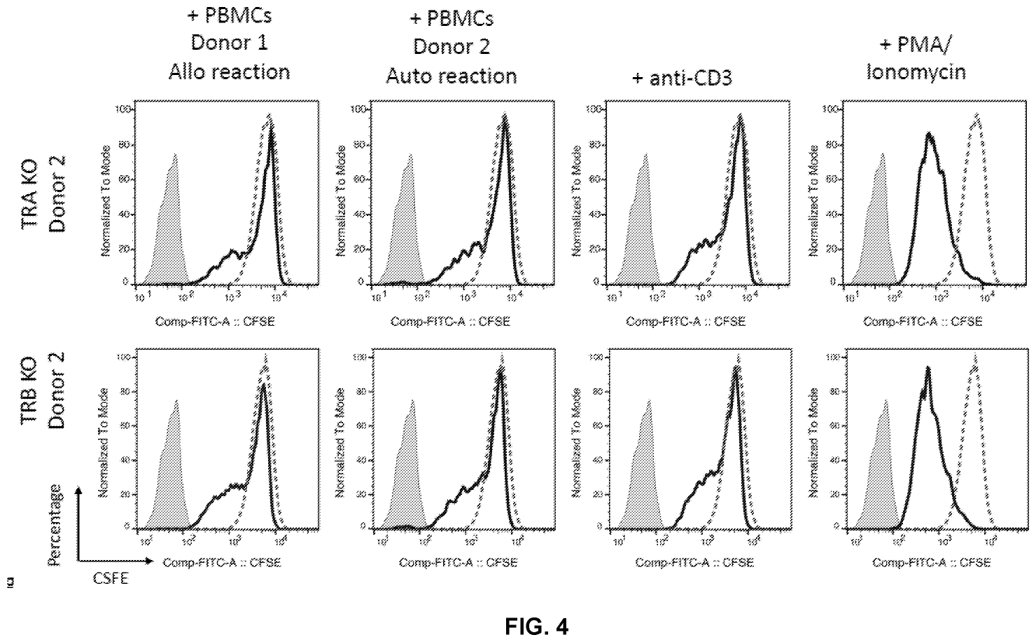

TCRαβ knock out (KO) cells were assessed for allogenicity by using a mixed lymphocyte reaction (MLR) assay. Carboxyfluorescein succinimidyl ester (CSFE) labeling dye was incorporated into TCRα and TCRβ KO T cells and cells were subsequently co-cultured at a 1:1 ratio with proliferation-arrested PBMCs (Streck, Inc.) from either matched (auto reaction) or mismatched (allo reaction) HLA donors (Donors 1 and 2, respectively). Phorbol myristate acetate (PMA) at 5 ng/mL and Ionomycin at 500 ng/mL was used as a positive control for independent TCR stimulation. Plate bound anti-CD3ε was also used as an indirect control to confirm the lack of TCR receptor in the TCRα and TCRβ knock out T cells. The proliferation of donor T cells was monitored by CSFE depletion; the basal levels of proliferation were measured following a twenty-four-hour incubation without stimulation, and levels were measured again following a five-day incubation period. CSFE dye dilutes by half upon cellular division and thus the amount of proliferation that occurred in the T cells was assessed and compared to matched and mismatched HLA donor controls. Results are shown in FIG. 4.

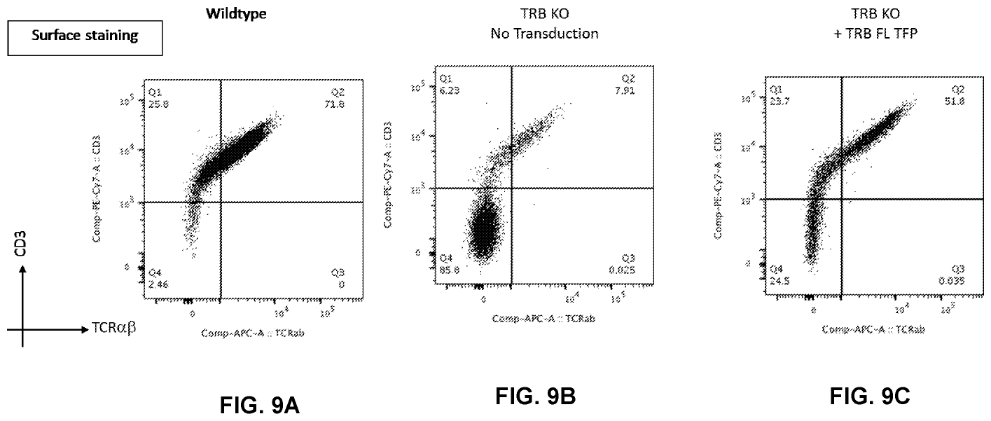

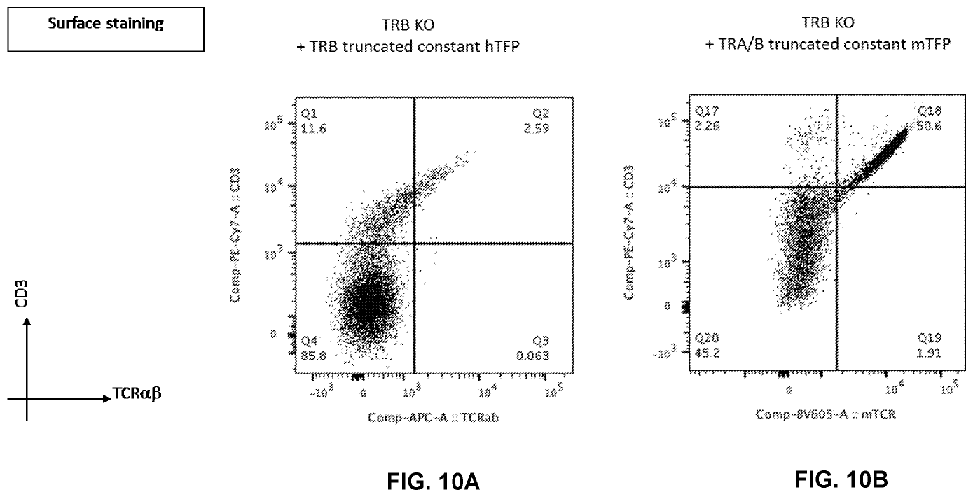

Surface expression of TCRαβ and CD3ε was analyzed as described in Example 2 for Jurkat T cells (FIGS. 9A-C) and donor T cells (FIGS. 10A-B). FIGS. 9A-C show surface expression of CD3 vs TCRαβ in wild type cells (FIG. 9A), TRB KO cells without transduction (FIG. 9B), TRB KO cells with transduction of TCRβ full length (FL) TFPs (FIG. 9C). The gates on the plots were drawn to delineate CD3 and TCRαβ negative-negative population of cells and the percentages of cells remaining in each quadrant are shown in the corners.

FIGS. 10A-B show surface expression of CD3 vs TCRαβ in TRB knockout cells transduced with a truncated human TRBC gene (FIG. 10A) and with a murine TRAC-T2A-TRBC gene (FIG. 10B). The gates on the plots were drawn to delineate CD3 and TCRαβ negative-negative population of cells and the percentages of cells remaining in each quadrant are shown in the corners.

### Example 5: T Cell Receptor Fusion Protein Expression in TCR Negative Cells

Inactivation of TRA or TRB blocks the translocation to the cell surface of all TCR subunits. Consequently, an exogenous TRA or TRB transgene is expressed in TRA^{-/-} or TRB^{-/-} cells, respectively, to get a functional TFP T cell.

### Transduction of Jurkat Cells

TFP transgenes were introduced in Jurkat cells using lentiviruses as described, e.g., in copending U.S. Patent Publication No. 2017-0166622. Jurkat cells were incubated with virus at a multiplicity of infection (MOI) of five. Medium was replaced twenty-four-hours post incubation. Transduction efficacy and TFP expression was assessed with flow cytometry using a ligand specific to the TFP binder of interest and/or surface expression of TCRαβ and CD3ε.

### Transduction of T Cells

TFP transgenes were introduced into T cells using lentiviruses as described, e.g., in copending U.S. Patent Publication No. 2017-0166622. T cells were centrifuged together with viruses at a multiplicity of infection (MOI) of five plus 5 μg/mL of polybrene during 100 minutes at 600 g. Medium was replaced twenty-four-hours post centrifugation. Transduction efficacy and TFP expression was assessed with flow cytometry using a ligand specific to the TFP binder of interest and/or surface expression of TCRαβ and CD3ε.

### Expression of Human TCR α/β TFP

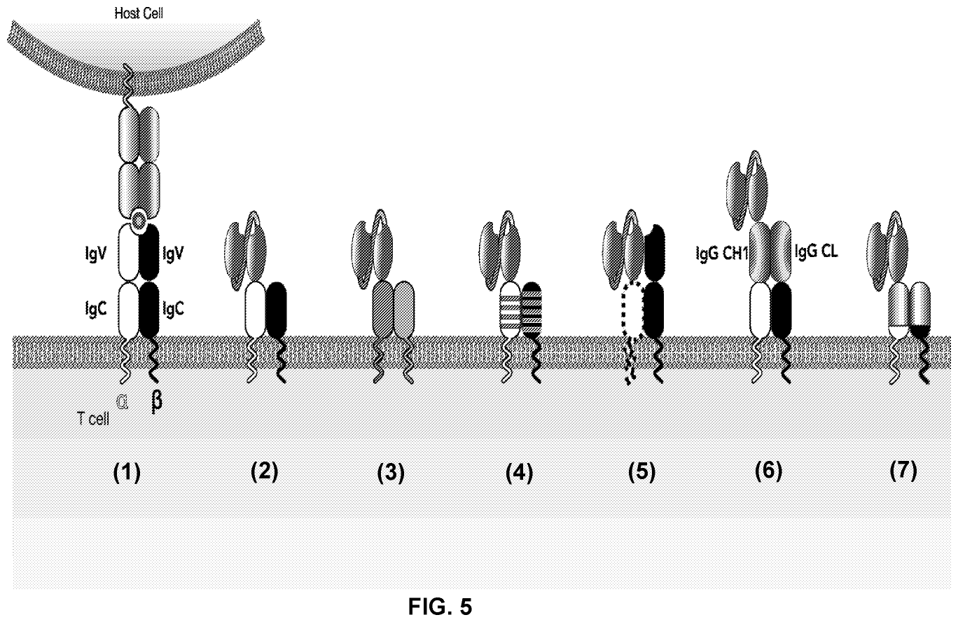

As TCRα negative cells still express TCRβ and, reciprocally, TCRα is expressed in TCRβ negative cells; therefore, TCRα TFPs were expressed in TRA^{-/-} cells and TCRβ TFPs were expressed in TRB^{-/-} cells. Multiple format of TCRα/β and TCRα/β TFPs were tested in TCR negative cells to determine the optimal construction to restore translocation of the entire TCR complex (FIG. 5). TCRα/β full length (FL) TFPs were generated by assembling any of the variable exons (V) with any of the junction exons (J) followed by all of the constant exons from TCR loci. In one embodiment, a diversity exon D could be placed between V and J. Possibly, mutation or indel could be added at the junction of each exon to mimic activity of recombination activating gene (RAG) enzymes. TRAV residues are numerated according to the international ImMunoGeneTics information system (IMGT, imgt.org).

TCRα_{(FL)} FMC63 TFP expressed in TRA^{-/-} cells

Nt-FMC63-TRA(V13-1₍₁₋₂₅₆₎; J13; C)-Ct

Nt-FMC63-TRA(V8-1; J20; C)-Ct

Nt-FMC63-TRA(V29DV5; J44; C)-Ct

Truncated TCRα TFP expressed TRA^{-/-} cells,

Nt-FMC63-TRA(V13-1₍₃₃₋₂₅₆₎; J13; C)-Ct

Nt-FMC63-TRA(V13-1₍₁₀₅₋₂₅₆₎; J13; C)-Ct

Truncated TCRα expressed TRA^{-/-} cells, TRAC residues are numerated according to the international ImMunoGeneTics information system (IMGT, www.imgt.org).

Nt-TRAC₇₋₁₇₄-Ct

Nt-TRAC₁₂₈₋₁₇₄-Ct

TCRβ_{(FL)} FMC63 TFP expressed in TRB^{-/-} cells

Nt-FMC63-TRB(V9; J1-1; C1)-Ct

Nt-FMC63-TRB(V7-9; J1-5; C1)-Ct

Nt-FMC63-TRB(V5-1; J2-2; C1)-Ct

Truncated TCRβ TFP expressed TRB^{-/-} cells, TRBC residues are numbered according to the international IMGT information system as noted above.

Nt-FMC63-TRBC1₍₋₈₎₋₁₇₃-Ct

Nt-FMC63-TRBC1₁₂₂₋₁₇₄-Ct

Nt-FMC63-TRBC1₁₂₇₋₁₇₄-Ct

### Expression of Truncated Human TCRα/β TFP

Overexpression of the constant domains of both TCRα and TCRβ may be sufficient to drive the translocation of the entire TCR complex to the cell surface. To test this, a TRP transgene was designed that encodes for the constant domains of TCRα and TCRβ separated by a 2A self-cleaving peptide. In one embodiment, the TFP binder is fused at the N terminal end of TRAC and/or TRBC. In another embodiment, the TFP is fused to a CD3 molecule and expressed independently of TR[A/B]C transgene.

### Expression of Truncated Murine TCRα/β TFP

Human TCR constant regions are interchangeable with their murine homologs. Additionally, mouse TCR constant regions increase stability of the CD3ζ/TCR complex when expressed in human cells. Consequently, a TFP transgene was designed that encodes the constant domains of mouse TCRα and TCRβ separated by a 2A self-cleaving peptide. In one embodiment, the TFP binder is fused at the N terminal end of mTRAC and/or mTRBC. In another embodiment, the TFP binder is carried by CD3 molecules and expressed independently of mTR[A/B]C transgene.

mTR[A/B]C transgenes express in TRA^{-/-} or TRB^{-/-} cells

Nt-FMC65-mTRAC₁₁₄₋₁₆₉-T2A-mTRBC₁₂₃₋₁₇₃-Ct

Nt-mTRAC₁₁₄₋₁₆₉-T2A-mTRBC₁₂₃₋₁₇₃-Ct

### Expression of Murinized Human TCRα/β TFP

To increase the affinity between the constant regions of human TCRα and TCRβ, a series of sequences were engineered wherein human TCR residues are replaced by mouse TCR. The substitutions were introduced in the constant region of TCRα, including residues P90S, E91D, S92V, S93P. The substitutions introduced in the constant region of TCRβ were E11K, S15A, F129I, E132A, Q135H. These substitutions made TRAC and TRBC sufficient for the translocation of the entire TCR complex to the cell surface. Therefore, the TFP is expressed through a transgene Nt-FMC63-TRAC₍₋₇₎₋₁₇₄ P90S, E91D, S92V, S93P-T2A-TRBC1₍₋₈₎₋₁₇₃ E11K, S15A, F129I, E132A, Q135H-Ct in TRA- or TRB^{-/-} cells.

### Expression of an Enhanced TCRα TFP

Several structure of the human TCRαβ complex are available in the protein data bank (PDB). Those structures highlight residues involve in TCRα/TCRβ interaction and other residues of TRAC close to TRBC but not involved in TCRα/TCRβ interaction. Hence, it is possible to enhance the affinity of TCRα for TCRβ by one or more of the following substitutions in TRAC: V22W, F85.5E, T84D, S85.1D, V84.1W,

Expression of enhanced TRAC-TFP in TRA^{-/-} cells restore translocation to the cell surface of the entire TCR. Enhanced TRAC-TFP in WT cells efficiently takes the place of endogenous TCRα molecule in the TCR complex. Enhance TRAC expresses without TFP binder efficiently restore translocation of TCR complex to the cell surface, in that case TFP binder is fuse to CD3 molecules and expressed independently of enhanced TRAC transgene or on the same transgene by placing a 2A self-cleaving peptide between both coding sequences (CDS).

Similarly, substitutions in TRBC enhance the interaction between TCRα/TCRβ. Substitutions V22W introduced individually or in combination in TRBC are sufficient to restore translocation to the cell surface of the entire TCR in TRB^{-/-} cells. Expression of enhanced TRBC-TFP in TRB^{-/-} cells restore translocation to the cell surface of the entire TCR. Expression of enhanced TRBC-TFP in wild type cells efficiently take the place of endogenous TCRβ molecule in the TCR complex. In that case of enhanced TRBC expresses without TFP binder the TFP binder is fused to CD3 molecules and expressed independently of enhanced TRBC transgene or on the same transgene by placing a 2A self-cleaving peptide between both CDS.

### Expression of a Hybrid IgG TCRα/β TFP

Interaction between TCRα and TCRβ is enhanced by replacing the variable domain of TCRα and TCRβ with IgG constant domains. Therefore, the IgG heavy chain constant domains CH1 was fused at the N terminal end of TRBC whereas the IgG light chain constant domains CL was fused at the N terminal end of TRAC. Finally, the TFP was added at the N terminal end of CL. In one embodiment, both constructs are encoded by the same transgene by placing a 2A self-cleaving peptide between them as indicated: Nt-FMC63-IgG_{CL(-7)-125}-TRAC₍₋₆₎₋₁₇₄-T2A-IgG_{CH1(-7)-122}-TRBC₍₋₈₎₋₁₇₃. In another embodiment, the position of IgG_{CL} and IgG_{CH1} is exchanged. In another embodiment, the TFP binder is fused at the N-terminal end of IgG_{CL} or/and IgG_{CH1} or fused to CD3 molecules and expressed independently. In another embodiment, residue substitutions are introduced to enhance CH1/CL interaction IgG_{CL}F7A, IgG_{CH1}A20L.

### Expression of Domain-Swapped TCR-TFP

TCRα/β/γ/δ molecules adopt a similar structural organization. At the N-terminal end, their V(D)J regions adopt an immunoglobulin (IgV) like conformation, whereas their C regions are constituted by an immunoglobulin (IgC) like domain followed by a connecting peptide (CP) a transmembrane domain (TM) and a short intracellular tail (IC) at the C-terminal end. Despite high structural homology between those molecules, TCRα only pairs with TCRβ and TCRγ only pairs with TCRδ. Consequently, swapping domain(s) of TCRα for TCRγ domain(s) and domain(s) of TCRβ for TCRγ domain(s) will generate TFPs that do not pair with endogenous TCR molecules. For instance, Nt-FMC63-IgCα-CPγ-TMγ-ICγ-2A-IgCβ-CPδ-ICδ-Ct produces an allogeneic receptor in which IgCαCPγTMγICγ specifically interacts with IgCbCPdICd and not with endogenous TCRβ in TRA^{-/-} cells or endogenous TCRα in TRB^{-/-} cells. In another embodiment, the TFP binder is fused at the N-terminal end of IgCβ or/and IgCα or fused to CD3 molecules and expressed independently. Different combinations of swapped domains may be used with the methods disclosed herein.

### Knock in (KI) a 2A Self-Cleaving Peptide in TCR Locus

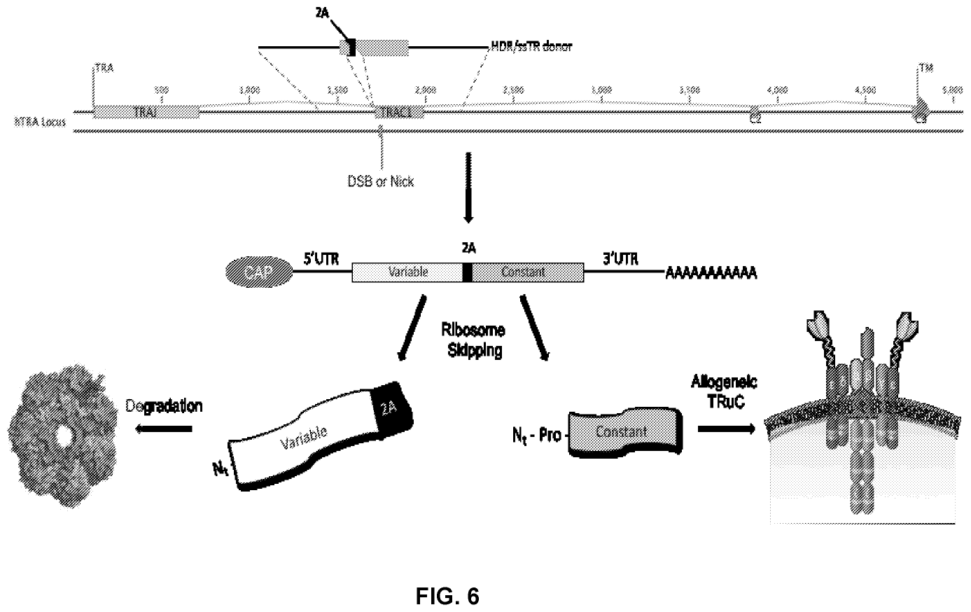

Introduction of a self-cleavage signal upstream of the CP domain in frame with TRAC or TRBC genes generates an endogenous truncated version of TCRα or TCRβ. Thus, the sequence downstream of the cleavage signal comprising the CP and TM domains is translocated to the cell surface; in contrast, the part upstream of the cleavage signal comprising the complementarity determining regions (CDRs) is not translocated to the cell surface. In one embodiment, the self-cleavage signal is inserted in frame in the TRAC or TRBC genes by homology-directed repair (HDR) or single stranded template repair (ssTR). HDR is induced by a DNA single-strand break (SSB) or a DNA double-strand break (DSB) whereas ssTR is induced by SSB only. In one embodiment, a custom endonuclease is used to generate a DSB upstream of CP region or a nickase to generate an SSB in the same area of TRAC or TRBC. Homologous donor DNA comprising a self-cleavage signal must have at least 40-base pair (bp) homology with the endogenous target, and can be single- or double-stranded, linear or circular. Additionally, homologous donor DNA comprises multiple base substitutions to not be cleaved by the custom endonuclease or nickase. In one embodiment, a CD3-TFP transgene is inserted into the cells prior or post gene editing. In another embodiment, the homologous donor DNA encodes the TFP sequence downstream of the self-cleavage peptide in frame with TRAC or TRBC. Consequently, the TFP-TCR fusion molecule is under control of the endogenous TCR receptor without risk of multiple random insertions of an exogenous promoter though the genome. A schematic is shown in FIG. 6.

### Example 6: Cytotoxicity of Human TCR-Negative T Cells Expressing TFPs

The luciferase-based cytotoxicity assay (“Luc-Cyto” assay) assesses the cytotoxicity of TFP T cells by indirectly measuring the luciferase enzymatic activity in the residual live target cells after co-culture.

### Generation of Firefly Luciferase (Luc) Expressing Tumor Cells

The target cells used in the Luc-Cyto assay were Nalm6-Luc (CD19 positive) and K562-Luc (CD19 negative were generated by stably transducing Nalm6 (DSMZ Cat. #ACC 128) and K562 ((ATCC® Cat. #CCL-243™)) cells to express firefly luciferase. The DNA encoding firefly luciferase was synthesized by GeneArt® (ThermoFisher) and inserted into the multiple cloning site of single-promoter lentiviral vector pCDH527A-1 (System Biosciences). The lentivirus was packaged according to manufacturer's instruction. Tumor cells were then transduced with the lentivirus for 24 hours and then selected with puromycin (5 μg/mL). The successful generation of Nalm6-Luc and K562-Luc cells was confirmed by measuring the luciferase enzymatic activity in the cells with Bright-Glo™ Luciferase Assay System (Promega).

### Phenotypic Characterization of Allo-TFP T Cells

Allogenic-TFP T cells were examined for their expression of human TCRαβ (with anti-human TCR, Miltenyi Bio, clone BW242/412), mouse TCRαβ (with anti-mouse TCRβ, BioLegend, clone H57-597), human CD3ε (with anti-human CD3ε BioLegend, clone UCHT1), human CD4 (with anti-human CD4, BioLegend, clone RPA-T4), human CD8 (with anti-human CD8, BioLegend, clone SK-1) and TFPs (with detection of the CD19 binder FMC63 by biotinylated CD19 (Cat. #CD9-H8259, AcroBio). Wild-type T cells (not edited) from the same donor were examined with the same panel as a comparison.

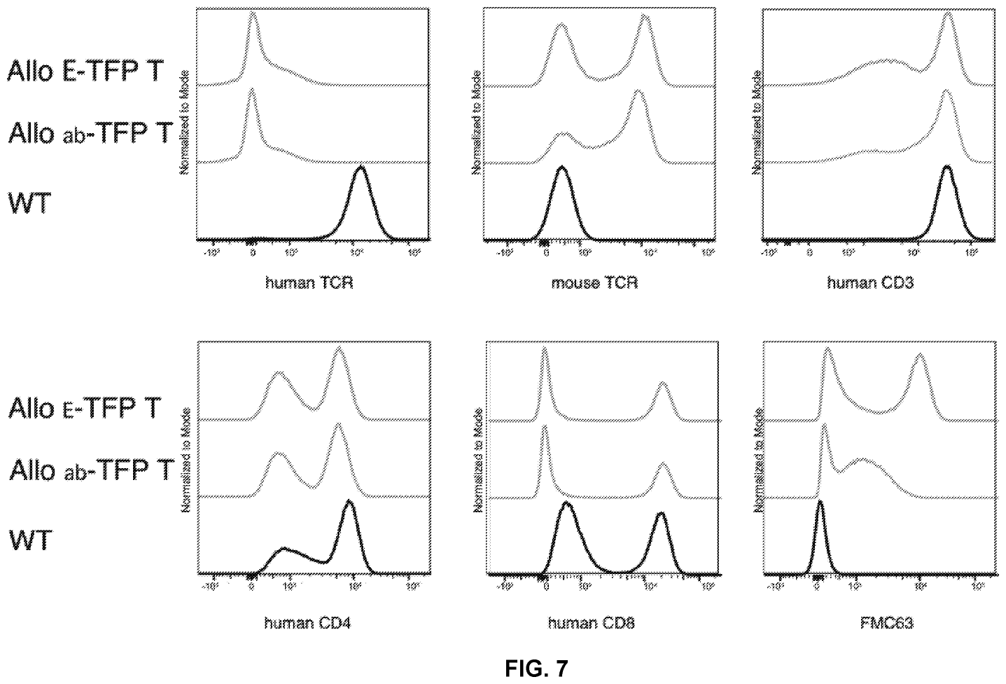

Results are shown in FIG. 7. Wild-type T cells show surface expression of human TCRαβ and CD3ε, but not mouse TCRβ. In contrast, allogenic TFP T cells show no surface expression of human TCRαβ, indicating successful editing. Surface expression of mouse TCRβ on allogenic TFP T cells is consistent with the detection of human CD3ε on the surface, suggesting successful re-assembly of the full TCR complex. Expression of human CD4 and CD8 are not significantly different between wild-type and TFP T cells. Detection of surface CD19 binder (FMC63, SEQ ID NO:X) is observed only for the allogenic TFP T cells.

### Luc-Cyto Assay Assessing the Cytotoxicity of T Cells

The Luc-Cyto assay was set up by mixing T cells with tumor cells at different effector (T cell) to target (tumor cell) (E-to-T) ratios. The target cells (Nalm6-Luc or K562-Luc) were plated at 10,000 cells per well in 96-well plates with RPMI-1640 medium supplemented with 10% heat-inactivated (HI) FBS. Allogeneic TFP T cells were added to the tumor cells at 30000, 10000, or 3333 cells per well to reach E-to-T ratios of 3-to-1, 1-to-1, or 1-to-3. The mixtures of cells were incubated for 24 hours at 37° C. with 5% CO₂. Luciferase enzymatic activity was measured using the Bright-Glo™ Luciferase Assay System (Promega), which measures activity from the residual live target cells in the T cell and tumor cell co-culture.

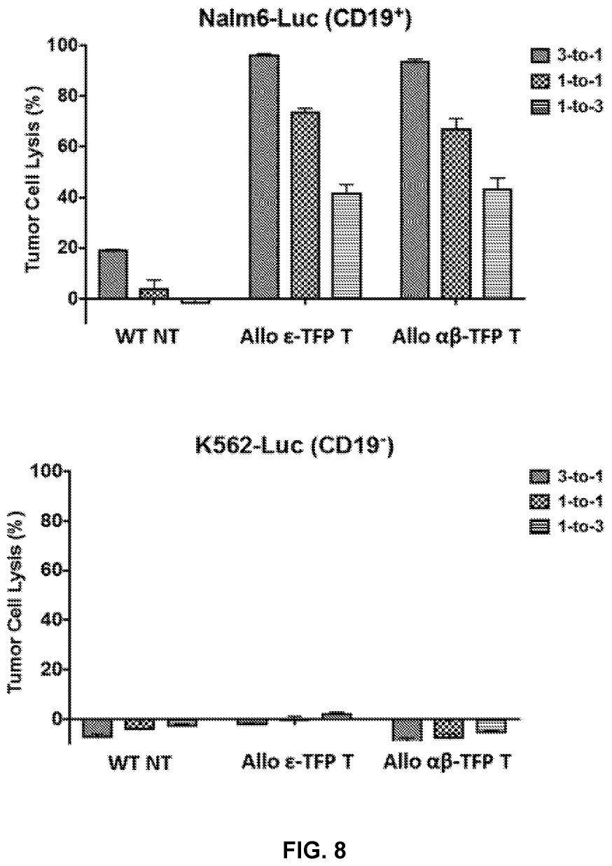

Results are shown in FIG. 8. The allogeneic TFP T cells, Allo CD3ε-TFP and Allo mTCRαβ-TFP T cells, showed robust and specific lysis against CD19 positive tumor cells Nalm6-Luc, but not the CD19 negative tumor cells K562-Luc.

### MLR of Human TCR-Negative T Cells Expressing TFPs

Human TCR-negative T cells expressing TFPs are assessed for allogenicity by using a mixed lymphocyte reaction (MLR) assay. Mismatched PBMC donor cells are first depleted of B cells by Magnetic-Activated Cell Sorting of CD19 negative cells. PBMCs are labelled with the lipophilic cellular labelling dye PKH and fixed with 0.4% paraformaldehyde. Simultaneously, a different colored PKH dye is incorporated into target T cells. Human TCR-negative T cells expressing TFPs and wild-type T cells from the same donor are subsequently co-cultured at either a 1:1 ratio (PBMCs to T cells) or T cells are cultured alone. The proliferation of donor T cells is monitored by tracking PKH dye over a six to twelve-day time point. PKH dye dilutes by half upon cellular division and thus the amount of proliferation that occurs in the T cells is assessed and compared to wild-type controls.

What is claimed is:

1. A recombinant nucleic acid comprising
(a) a sequence encoding a T cell receptor (TCR) fusion protein (TFP) comprising
(i) a TCR subunit comprising
(1) at least a portion of a TCR extracellular domain,
(2) a transmembrane domain, and
(3) an intracellular domain comprising a stimulatory domain from an intracellular signaling domain of CD3 epsilon, CD3 gamma, CD3 delta, TCR alpha or TCR beta, and
(ii) a human or humanized antibody comprising an antigen binding domain; and
(b) a sequence encoding a TCR constant domain, wherein the TCR constant domain is a TCR alpha constant domain, a TCR beta constant domain or a TCR alpha constant domain and a TCR beta constant domain;
wherein the TCR subunit and the antibody are operatively linked, and
wherein the TFP functionally incorporates into a TCR complex when expressed in a modified T cell comprising a functional disruption of an endogenous TCR.

2. A recombinant nucleic acid comprising
(a) a sequence encoding a T cell receptor (TCR) fusion protein (TFP) comprising
(i) a TCR subunit comprising
(1) at least a portion of a TCR extracellular domain,
(2) a transmembrane domain, and
(3) an intracellular domain comprising a stimulatory domain from an intracellular signaling domain of CD3 epsilon, CD3 gamma, CD3 delta, TCR alpha or TCR beta, and
(ii) a binding ligand or a fragment thereof that is capable of binding to an antibody or fragment thereof, and
(b) a sequence encoding a TCR constant domain, wherein the TCR constant domain is a TCR alpha constant domain, a TCR beta constant domain or a TCR alpha constant domain and a TCR beta constant domain;
wherein the TCR subunit and the binding ligand or fragment thereof are operatively linked, and
wherein the TFP functionally incorporates into TCR complex when expressed in a modified T cell comprising a functional disruption of an endogenous TCR.

3. The recombinant nucleic acid of claim 2, wherein the binding ligand is capable of binding an Fc domain of the antibody.

4. The recombinant nucleic acid of claim 2, wherein the binding ligand is capable of selectively binding an IgG1 antibody.

5. The recombinant nucleic acid of claim 2, wherein the binding ligand is capable of specifically binding an IgG4 antibody.

6. The recombinant nucleic acid of claim 2, wherein the antibody or fragment thereof binds to a cell surface antigen.

7. The recombinant nucleic acid of claim 2, wherein the antibody or fragment thereof binds to a cell surface antigen on the surface of a tumor cell.

8. The recombinant nucleic acid of claim 2, wherein the binding ligand comprises a monomer, a dimer, a trimer, a tetramer, a pentamer, a hexamer, a heptamer, an octomer, a nonamer, or a decamer.

9. The recombinant nucleic acid of claim 2, wherein the binding ligand does not comprise an antibody or fragment thereof.

10. The recombinant nucleic acid of claim 9, wherein the binding ligand comprises a CD16 polypeptide or fragment thereof.

11. The recombinant nucleic acid of claim 10, wherein the binding ligand comprises a CD16-binding polypeptide.

12. The recombinant nucleic acid of claim 2, wherein the binding ligand is human or humanized.

13. The recombinant nucleic acid of claim 2, further comprising a nucleic acid sequence encoding an antibody or fragment thereof capable of being bound by the binding ligand.

14. The recombinant nucleic acid of claim 13, wherein the antibody or fragment thereof is capable of being secreted from a cell.

15. A recombinant nucleic acid comprising
(a) a sequence encoding a T cell receptor (TCR) fusion protein (TFP) comprising
(i) a TCR subunit comprising
(1) at least a portion of a TCR extracellular domain,
(2) a transmembrane domain, and
(3) an intracellular domain comprising a stimulatory domain from an intracellular signaling domain of CD3 epsilon, CD3 gamma, CD3 delta, TCR alpha or TCR beta, and
(ii) an antigen domain comprising a ligand or a fragment thereof that binds to a receptor or polypeptide expressed on a surface of a cell; and
(b) a sequence encoding a TCR constant domain, wherein the TCR constant domain is a TCR alpha constant domain, a TCR beta constant domain or a TCR alpha constant domain and a TCR beta constant domain;
wherein the TCR subunit and the antigen domain are operatively linked, and
wherein the TFP functionally incorporates into a TCR complex when expressed in a modified T cell comprising a functional disruption of an endogenous TCR.

16. The recombinant nucleic acid of claim 15, wherein the antigen domain comprises a ligand.

17. The recombinant nucleic acid of claim 15, wherein the ligand binds to the receptor of a cell.

18. The recombinant nucleic acid of claim 15, wherein the ligand binds to the polypeptide expressed on a surface of a cell.

19. The recombinant nucleic acid of claim 15, wherein the receptor or polypeptide expressed on a surface of a cell comprises a stress response receptor or polypeptide.

20. The recombinant nucleic acid of claim 15, wherein the receptor or polypeptide expressed on a surface of a cell is an MHC class I-related glycoprotein.

21. The recombinant nucleic acid of claim 20, wherein the MHC class I-related glycoprotein is selected from the group consisting of MICA, MICB, RAETIE, RAET1G, ULBP1, ULBP2, ULBP3, ULBP4 and combinations thereof.

22. The recombinant nucleic acid of claim 15, wherein the antigen domain comprises a monomer, a dimer, a trimer, a tetramer, a pentamer, a hexamer, a heptamer, an octomer, a nonamer, or a decamer.

23. The recombinant nucleic acid of claim 22, wherein the antigen domain comprises a monomer or a dimer of the ligand or fragment thereof.

24. The recombinant nucleic acid of claim 15, wherein the ligand or fragment thereof is a monomer, a dimer, a trimer, a tetramer, a pentamer, a hexamer, a heptamer, an octomer, a nonamer, or a decamer.

25. The recombinant nucleic acid of claim 24, wherein the ligand or fragment thereof is a monomer or a dimer.

26. The recombinant nucleic acid of claim 15, wherein the antigen domain does not comprise an antibody or fragment thereof.

27. The recombinant nucleic acid of claim 15, wherein the antigen domain does not comprise a variable region.

28. The recombinant nucleic acid of claim 15, wherein the antigen domain does not comprise a CDR.

29. The recombinant nucleic acid of claim 15, wherein the ligand or fragment thereof is a Natural Killer Group 2D (NKG2D) ligand or a fragment thereof.

30. The recombinant nucleic acid of any one of claims 1-29, wherein the TCR constant domain incorporates into a functional TCR complex when expressed in a T cell.

31. The recombinant nucleic acid of any one of claims 1-30, wherein the TCR constant domain incorporates into a same functional TCR complex as the functional TCR complex that incorporates the TFP when expressed in a T cell.

32. The recombinant nucleic acid of any one of claims 1-31, wherein the sequence encoding the TFP and the sequence encoding the TCR constant domain are contained within a same nucleic acid molecule.

33. The recombinant nucleic acid of any one of claims 1-31, wherein the sequence encoding the TFP and the sequence encoding the TCR constant domain are contained within different nucleic acid molecules.

34. The recombinant nucleic acid of claim 1-33, wherein the TCR subunit and the antibody domain, the antigen domain or the binding ligand or fragment thereof are operatively linked by a linker sequence.

35. The recombinant nucleic acid of claim 34, wherein the linker sequence comprises (G₄S)ₙ, wherein n=1 to 4.

36. The recombinant nucleic acid of any one of claims 1-35, wherein the transmembrane domain is a TCR transmembrane domain from CD3 epsilon, CD3 gamma, CD3 delta, TCR alpha or TCR beta.

37. The recombinant nucleic acid of any one of claims 1-36, wherein the intracellular domain is derived from only CD3 epsilon, only CD3 gamma, only CD3 delta, only TCR alpha or only TCR beta.

38. The recombinant nucleic acid of any one of claims 1-37, wherein the TCR subunit comprises (i) at least a portion of a TCR extracellular domain, (ii) a TCR transmembrane domain, and (iii) a TCR intracellular domain, wherein at least two of (i), (ii), and (iii) are from the same TCR subunit.

39. The recombinant nucleic acid of any one of claims 1-38, wherein the TCR extracellular domain comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications.

40. The recombinant nucleic acid of any one of claims 1-39, wherein the TCR subunit comprises a transmembrane domain comprising a transmembrane domain of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

41. The recombinant nucleic acid of any one of claims 1-40, wherein the TCR subunit comprises a TCR intracellular domain comprising a stimulatory domain of a protein selected from an intracellular signaling domain of CD3 epsilon, CD3 gamma or CD3 delta, or an amino acid sequence having at least one modification thereto.

42. The recombinant nucleic acid of any one of claims 1-41, wherein the TCR subunit comprises an intracellular domain comprising a stimulatory domain of a protein selected from a functional signaling domain of 4-1BB and/or a functional signaling domain of CD3 zeta, or an amino acid sequence having at least one modification thereto.

43. The recombinant nucleic acid of any one of claims 1-42, further comprising a sequence encoding a costimulatory domain.

44. The recombinant nucleic acid of claim 43, wherein the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137), and amino acid sequences thereof having at least one but not more than 20 modifications thereto.

45. The recombinant nucleic acid of any one of claims 1-44, wherein the TCR subunit comprises an immunoreceptor tyrosine-based activation motif (ITAM) of a TCR subunit that comprises an ITAM or portion thereof of a protein selected from the group consisting of CD3 zeta TCR subunit, CD3 epsilon TCR subunit, CD3 gamma TCR subunit, CD3 delta TCR subunit, TCR zeta chain, Fc epsilon receptor 1 chain, Fc epsilon receptor 2 chain, Fc gamma receptor 1 chain, Fc gamma receptor 2a chain, Fc gamma receptor 2b1 chain, Fc gamma receptor 2b2 chain, Fc gamma receptor 3a chain, Fc gamma receptor 3b chain, Fc beta receptor 1 chain, TYROBP (DAP12), CD5, CD16a, CD16b, CD22, CD23, CD32, CD64, CD79α, CD79b, CD89, CD278, CD66d, functional fragments thereof, and amino acid sequences thereof having at least one but not more than 20 modifications thereto.

46. The recombinant nucleic acid of claim 45, wherein the ITAM replaces an ITAM of CD3 gamma, CD3 delta, or CD3 epsilon.

47. The recombinant nucleic acid of claim 45, wherein the ITAM is selected from the group consisting of CD3 zeta TCR subunit, CD3 epsilon TCR subunit, CD3 gamma TCR subunit, and CD3 delta TCR subunit and replaces a different ITAM selected from the group consisting of CD3 zeta TCR subunit, CD3 epsilon TCR subunit, CD3 gamma TCR subunit, and CD3 delta TCR subunit.

48. The recombinant nucleic acid of any one of claims 1-47, wherein the TFP, the TCR alpha constant domain, the TCR beta constant domain, and any combination thereof is capable of functionally interacting with an endogenous TCR complex and/or at least one endogenous TCR polypeptide.

49. The recombinant nucleic acid of any one of claims 1-48, wherein
(a) the TCR constant domain is a TCR alpha constant domain and the TFP functionally integrates into a TCR complex comprising an endogenous subunit of TCR beta, CD3 epsilon, CD3 gamma, CD3 delta, or a combination thereof,
(b) the TCR constant domain is a TCR beta constant domain and the TFP functionally integrates into a TCR complex comprising an endogenous subunit of TCR alpha, CD3 epsilon, CD3 gamma, CD3 delta, or a combination thereof; or
(c) the TCR constant domain is a TCR alpha constant domain and a TCR beta constant domain and the TFP functionally integrates into a TCR complex comprising an endogenous subunit of CD3 epsilon, CD3 gamma, CD3 delta, or a combination thereof.

50. The recombinant nucleic acid of any one of claims 1-49, wherein the at least one but not more than 20 modifications thereto comprise a modification of an amino acid that mediates cell signaling or a modification of an amino acid that is phosphorylated in response to a ligand binding to the TFP.

51. The recombinant nucleic acid of any one of claims 1 and 34-50, wherein the human or humanized antibody is an antibody fragment.

52. The recombinant nucleic acid of claim 51, wherein the antibody fragment is a scFv, a single domain antibody domain, a V_{H} domain or a V_{L} domain.

53. The recombinant nucleic acid of any one of claims 1 and 34-52, wherein an antigen binding domain is selected from a group consisting of an anti-CD19 binding domain, anti-B-cell maturation antigen (BCMA) binding domain, anti-mesothelin (MSLN) binding domain, an anti-IL13Rα2 binding domain, an anti-MUC16 binding domain, an anti-CD22 binding domain, an anti-PD-1 binding domain, an anti BAFF or BAFF receptor binding domain, and anti-ROR-1 binding domain.

54. The recombinant nucleic acid of any one of claims 1-53, wherein the nucleic acid is selected from the group consisting of a DNA and an RNA.

55. The recombinant nucleic acid of any one of claims 1-54, wherein the nucleic acid is an mRNA.

56. The recombinant nucleic acid of any one of claims 1-55, wherein the recombinant nucleic acid comprises a nucleic acid analog, wherein the nucleic acid analog is not in an encoding sequence of the recombinant nucleic acid.

57. The recombinant nucleic acid of claim 56, wherein the nucleic analog is selected from the group consisting of 2′-O-methyl, 2′-O-methoxyethyl (2′-O-MOE), 2′-O-aminopropyl, 2′-deoxy, T-deoxy-2′-fluoro, 2′-O-aminopropyl (2′-O-AP), 2′-O-dimethylaminoethyl (2′-O-DMAOE), 2′-O-dimethylaminopropyl (2′-O-DMAP), T-O-dimethylaminoethyloxyethyl (2′-O-DMAEOE), 2′-O—N-methylacetamido (2′-O-NMA) modified, a locked nucleic acid (LNA), an ethylene nucleic acid (ENA), a peptide nucleic acid (PNA), a 1′,5′-anhydrohexitol nucleic acid (HNA), a morpholino, a methylphosphonate nucleotide, a thiolphosphonate nucleotide, and a 2′-fluoro N3-P5′-phosphoramidite.

58. The recombinant nucleic acid of any one of claims 1-57, further comprising a leader sequence.

59. The recombinant nucleic acid of any one of claims 1-58, further comprising a promoter sequence.

60. The recombinant nucleic acid of any one of claims 1-59, further comprising a sequence encoding a poly(A) tail.

61. The recombinant nucleic acid of any one of claims 1-60, further comprising a 3′UTR sequence.

62. The recombinant nucleic acid of any one of claims 1-61, wherein the nucleic acid is an isolated nucleic acid or a non-naturally occurring nucleic acid.

63. The recombinant nucleic acid molecule of any one of claims 1-62, wherein the nucleic acid is an in vitro transcribed nucleic acid.

64. The recombinant nucleic acid molecule of any one of claims 1-63, further comprising a sequence encoding a TCR alpha transmembrane domain.

65. The recombinant nucleic acid molecule of any one of claims 1-63, further comprising a sequence encoding a TCR beta transmembrane domain.

66. The recombinant nucleic acid of any one of claims 1-63, further comprising a sequence encoding a TCR alpha transmembrane domain and a sequence encoding a TCR beta transmembrane domain.

67. A vector comprising the recombinant nucleic acid of any one of claims 1-66.

68. The vector of claim 67, wherein the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector.

69. The vector of claim 67 or 68, wherein the vector is an AAV6 vector.

70. The vector of any one of claims 67-69, further comprising a promoter.

71. The vector of any one of claims 67-70, wherein the vector is an in vitro transcribed vector.

72. A modified T cell comprising the recombinant nucleic acid of any one of claims 1-66, or the vector of any one of claims 67-71, wherein the modified T cell comprises a functional disruption of an endogenous TCR.

73. A modified T cell comprising the sequence encoding the TFP of the nucleic acid of any one of claims 1-66 or a TFP encoded by the sequence of the nucleic acid of any one of claims 1-66 encoding the TFP, wherein the modified T cell comprises a functional disruption of an endogenous TCR.

74. A modified allogenic T cell comprising the sequence encoding the TFP of any one of claims 1-66 or a TFP encoded by the sequence of the nucleic acid of any one of claims 1-66 encoding the TFP.

75. The modified T cell of any one of claims 72-74, wherein the T cell further comprises a heterologous sequence encoding a TCR constant domain, wherein the TCR constant domain is a TCR alpha constant domain, a TCR beta constant domain or a TCR alpha constant domain and a TCR beta constant domain.

76. The modified T cell of any one of claims 72-75, wherein the endogenous TCR that is functionally disrupted is an endogenous TCR alpha chain, an endogenous TCR beta chain, or an endogenous TCR alpha chain and an endogenous TCR beta chain.

77. The modified T cell of any one of claims 72-76, wherein the endogenous TCR that is functionally disrupted has reduced binding to MHC-peptide complex compared to that of an unmodified control T cell.

78. The modified T cell of any one of claims 72-77, wherein the functional disruption is a disruption of a gene encoding the endogenous TCR.

79. The modified T cell of claim 78, wherein the disruption of a gene encoding the endogenous TCR is a removal of a sequence of the gene encoding the endogenous TCR from the genome of a T cell.

80. The modified T cell of any one of claims 72-79, wherein the T cell is a human T cell selected from CD4 cells, CD8 cells, naive T-cells, memory stem T-cells, central memory T-cells, double negative T-cells, effector memory T-cells, effector T-cells, ThO cells, TcO cells, Th1 cells, Tc1 cells, Th2 cells, Tc2 cells, Th17 cells, Th22 cells, gamma/delta T-cells, natural killer (NK) cells, natural killer T (NKT) cells, hematopoietic stem cells and pluripotent stem cells.

81. The modified T cell of any one of claims 72-80, wherein the T cell is a CD8+ or CD4+ T cell.

82. The modified T cell of any one of claims 72-81, wherein the T cell is an allogenic T cell.

83. The modified T cell of any one of claims 72-82, further comprising a nucleic acid encoding an inhibitory molecule that comprises a first polypeptide comprising at least a portion of an inhibitory molecule, associated with a second polypeptide comprising a positive signal from an intracellular signaling domain.

84. The modified T cell of claim 83, wherein the inhibitory molecule comprises the first polypeptide comprising at least a portion of PD1 and the second polypeptide comprising a costimulatory domain and primary signaling domain.

85. A pharmaceutical composition comprising:
(a) the modified T cells of any one of claims 72-84; and
(b) a pharmaceutically acceptable carrier.

86. A method of producing the modified T cell of any one of claims 72-84, the method comprising
(a) disrupting an endogenous TCR gene encoding a TCR alpha chain, a TCR beta chain, or a TCR alpha chain and a TCR beta chain; thereby producing a T cell containing a functional disruption of an endogenous TCR gene; and
(b) transducing the T cell containing a functional disruption of an endogenous TCR gene with the recombinant nucleic acid of any one of claims 1-63, or the vector of any one of claims 67-71.

87. The method of claim 86, wherein disrupting comprises transducing the T cell with a nuclease protein or a nucleic acid sequence encoding a nuclease protein that targets the endogenous gene encoding a TCR alpha chain, a TCR beta chain, or a TCR alpha chain and a TCR beta chain.

88. A method of producing the modified T cell of any one of claims 72-84, the method comprising transducing a T cell containing a functional disruption of an endogenous TCR gene with the recombinant nucleic acid of any one of claims 1-63, or the vector of any one of claims 67-71.

89. The method of claim 88, wherein the T cell containing a functional disruption of an endogenous TCR gene is a T cell containing a functional disruption of an endogenous TCR gene encoding a TCR alpha chain, a TCR beta chain, or a TCR alpha chain and a TCR beta chain.

90. The method of any one of claims 86-89, wherein the T cell is a human T cell.

91. The method of any one of claims 86-90, wherein the T cell containing a functional disruption of an endogenous TCR gene has reduced binding to MHC-peptide complex compared to that of an unmodified control T cell.

92. The method of any one of claims 86-91, wherein the nuclease is a meganuclease, a zinc-finger nuclease (ZFN), a transcription activator-like effector nuclease (TALEN), a CRISPR/Cas nuclease, or a megaTAL nuclease.

93. The method of any one of claims 86-92, wherein the sequence comprised by the recombinant nucleic acid or the vector is inserted into the endogenous TCR subunit gene at the cleavage site, and wherein the insertion of the sequence into the endogenous TCR subunit gene functionally disrupts the endogenous TCR subunit.

94. The method of any one of claims 86-93, wherein the nuclease is a meganuclease.

95. The method of claim 94, wherein the meganuclease comprises a first subunit and a second subunit, wherein the first subunit binds to a first recognition half-site of the recognition sequence, and wherein the second subunit binds to a second recognition half-site of the recognition sequence.

96. The method of claim 95, wherein the meganuclease is a single-chain meganuclease comprising a linker, wherein the linker covalently joins the first subunit and the second subunit.

97. A method of treating cancer in a subject in need thereof, the method comprising administering to the subject a therapeutically effective amount of the pharmaceutical composition of claim 85.

98. A method of treating cancer in a subject in need thereof, the method comprising administering to the subject a pharmaceutical composition comprising (a) a modified T cell produced according to the method of any one of claims 86-96; and (b) a pharmaceutically acceptable carrier.

99. A method of treating cancer in a subject in need thereof, the method comprising administering to the subject a pharmaceutical composition comprising (a) a modified T cell produced according to the method of any one of claims 88-96; and (b) a pharmaceutically acceptable carrier.

100. The method of any one of claims 97-99, wherein the modified T cell is an allogeneic T cell.

101. The method of any one of claims 97-100, wherein less cytokines are released in the subject compared a subject administered an effective amount of an unmodified control T cell.

102. The method of any one of claims 97-101, wherein less cytokines are released in the subject compared a subject administered an effective amount of a modified T cell comprising the recombinant nucleic acid of any one of claims 1-66, or the vector of any one of claims 67-71.

103. The method of any one of claims 97-102, wherein the method comprises administering the pharmaceutical composition in combination with an agent that increases the efficacy of the pharmaceutical composition.

104. The method of any one of claims 97-103, wherein the method comprises administering the pharmaceutical composition in combination with an agent that ameliorates one or more side effects associated with the pharmaceutical composition.

105. The method of any one of claims 97-104, wherein the cancer is a solid cancer, a lymphoma or a leukemia.

106. The method of any one of claims 97-105, wherein the cancer is selected from the group consisting of renal cell carcinoma, breast cancer, lung cancer, ovarian cancer, prostate cancer, colon cancer, cervical cancer, brain cancer, liver cancer, pancreatic cancer, kidney and stomach cancer.

107. The recombinant nucleic acid of any one of claims 1-66, the vector of any one of claims 67-71, the modified T cell of any one of claims 72-84, or the pharmaceutical composition of claim 85, for use as a medicament or in the preparation of a medicament.


SEQUENCE LISTING (93 sequences)

SEQ ID NO: 1 - protein, 20 aa, Artificial Sequence
  Description of Artificial Sequence Synthetic peptide
  MISC_FEATURE (1)..(20): This sequence may encompass 1-4 ′Gly Gly Gly Gly Ser′ repeating units
GGGGSGGGGSGGGGSGGGGS

SEQ ID NO: 2 - protein, 4 aa, Artificial Sequence
  Description of Artificial Sequence Synthetic peptide
GGGS

SEQ ID NO: 3 - protein, 10 aa, Artificial Sequence
  Description of Artificial Sequence Synthetic peptide
GGGGSGGGGS

SEQ ID NO: 4 - DNA, 30 bp, Artificial Sequence
  Description of Artificial Sequence Synthetic oligonucleotide
ggtggcggaggttctggaggtggaggttcc

SEQ ID NO: 5 - protein, 20 aa, Artificial Sequence
  Description of Artificial Sequence Synthetic peptide
GGGGSGGGGSGGGGSGGGGS

SEQ ID NO: 6 - protein, 15 aa, Artificial Sequence
  Description of Artificial Sequence Synthetic peptide
GGGGSGGGGSGGGGS

SEQ ID NO: 7 - protein, 4 aa, Artificial Sequence
  Description of Artificial Sequence Synthetic peptide
GGGS

SEQ ID NO: 8 - protein, 20 aa, Artificial Sequence
  Description of Artificial Sequence Synthetic peptide
  MISC_FEATURE (1)..(20): This sequence may encompass 2-4 ′Gly Gly Gly Gly Ser′ repeating units
GGGGSGGGGSGGGGSGGGGS

SEQ ID NO: 9 - protein, 15 aa, Artificial Sequence
  Description of Artificial Sequence Synthetic peptide
  MISC_FEATURE (1)..(15): This sequence may encompass 1-3 ′Gly Gly Gly Gly Ser′ repeating units
GGGGSGGGGSGGGGS

SEQ ID NO: 10 - RNA, 5000 nt, Artificial Sequence
  Description of Artificial Sequence Synthetic polynucleotide
  misc_feature (1)..(5000): This sequence may encompass 50-5000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - protein, 30 aa, Artificial Sequence
  Description of Artificial Sequence Synthetic polypeptide
  MISC_FEATURE (1)..(30): This sequence may encompass 1-6 ′Gly Gly Gly Gly Ser′ repeating units
GGGGSGGGGSGGGGSGGGGSGGGGSGGGGS

SEQ ID NO: 12 - protein, 5 aa, Artificial Sequence
  Description of Artificial Sequence Synthetic peptide
GGGGS

SEQ ID NO: 13 - RNA, 2000 nt, Artificial Sequence
  Description of Artificial Sequence Synthetic polynucleotide
  misc_feature (1)..(2000): This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 100 bp, Artificial Sequence
  Description of Artificial Sequence Synthetic polynucleotide
tttttttttttttttttttttttttttttttttttttttttttttttttttttttttttttttttttttttttttttttttttttttttttttttttttt

SEQ ID NO: 15 - DNA, 5000 bp, Artificial Sequence
  Description of Artificial Sequence Synthetic polynucleotide
  misc_feature (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 16 - RNA, 5000 nt, Artificial Sequence; identical to SEQ ID NO: 10
  Description of Artificial Sequence Synthetic polynucleotide
  misc_feature (1)..(5000): This sequence may encompass 100-5000 nucleotides

SEQ ID NO: 17 - RNA, 400 nt, Artificial Sequence
  Description of Artificial Sequence Synthetic polynucleotide
  misc_feature (1)..(400): This sequence may encompass 100-400 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 20 bp, Artificial Sequence
  Description of Artificial Sequence Synthetic oligonucleotide
tctctcagctggtacacggc

SEQ ID NO: 19 - DNA, 20 bp, Artificial Sequence
  Description of Artificial Sequence Synthetic oligonucleotide
ctcgaccagcttgacatcac

SEQ ID NO: 20 - DNA, 20 bp, Artificial Sequence
  Description of Artificial Sequence Synthetic oligonucleotide
gattaaacccggccactttc

SEQ ID NO: 21 - DNA, 23 bp, Homo sapiens
tccctcagctggtacaaggatgg

SEQ ID NO: 22 - DNA, 23 bp, Homo sapiens
tctgtcaactggtacatggcaag

SEQ ID NO: 23 - DNA, 23 bp, Homo sapiens
tctcatagctggtacatggcggg

SEQ ID NO: 24 - DNA, 23 bp, Homo sapiens
tttctcagctggtacatggaggg

SEQ ID NO: 25 - DNA, 23 bp, Homo sapiens
gcactcagctggtacccggcaag

SEQ ID NO: 26 - DNA, 23 bp, Homo sapiens
tcactcagctggtacatgggcag

SEQ ID NO: 27 - DNA, 23 bp, Homo sapiens
tctcccagctgggacacggtgag

SEQ ID NO: 28 - DNA, 23 bp, Homo sapiens
tcaatcagctggtgcacggctgg

SEQ ID NO: 29 - DNA, 23 bp, Homo sapiens
tctcacagctgatatacggctgg

SEQ ID NO: 30 - DNA, 23 bp, Homo sapiens
ctccaccaccttgacctcaccgg

SEQ ID NO: 31 - DNA, 23 bp, Homo sapiens
ctcaaccagaatgacatcaccag

SEQ ID NO: 32 - DNA, 23 bp, Homo sapiens
ctagaccagcttgacctccccag

SEQ ID NO: 33 - DNA, 23 bp, Homo sapiens
ctagaccagcttggcaacacagg

SEQ ID NO: 34 - DNA, 23 bp, Homo sapiens
gaataaaaccggccactttgggg

SEQ ID NO: 35 - DNA, 23 bp, Homo sapiens
gattatacctggccacattcaag

SEQ ID NO: 36 - DNA, 20 bp, Artificial Sequence
  Description of Artificial Sequence Synthetic oligonucleotide
acactggtgtgcctggccac

SEQ ID NO: 37 - DNA, 20 bp, Artificial Sequence
  Description of Artificial Sequence Synthetic oligonucleotide
agggcgggctgctccttgag

SEQ ID NO: 38 - DNA, 20 bp, Artificial Sequence
  Description of Artificial Sequence Synthetic oligonucleotide
ctgcctgagcagccgcctga

SEQ ID NO: 39 - DNA, 20 bp, Artificial Sequence
  Description of Artificial Sequence Synthetic oligonucleotide
gcgggggttctgccagaagg

SEQ ID NO: 40 - DNA, 23 bp, Homo sapiens
actctgggctgcctggccacggg

SEQ ID NO: 41 - DNA, 23 bp, Homo sapiens
actctgttgtgcctggacaccgg

SEQ ID NO: 42 - DNA, 23 bp, Homo sapiens
tcacaggtgagcctggccacagg

SEQ ID NO: 43 - DNA, 23 bp, Homo sapiens
gcacgggtgggcctggccactgg

SEQ ID NO: 44 - DNA, 23 bp, Homo sapiens
gcaggggtgtgcctggccactgg

SEQ ID NO: 45 - DNA, 23 bp, Homo sapiens
atcctgctgtgcctggccacagg

SEQ ID NO: 46 - DNA, 23 bp, Homo sapiens
tctctggtgtgcctggccaagag

SEQ ID NO: 47 - DNA, 23 bp, Homo sapiens
acacatgtgggcctggccacggg

SEQ ID NO: 48 - DNA, 23 bp, Homo sapiens
agcctggtgtgtctggccactgg

SEQ ID NO: 49 - DNA, 23 bp, Homo sapiens
cctctggtgtgcctggccccagg

SEQ ID NO: 50 - DNA, 23 bp, Homo sapiens
ccacttgtgtgcatggccactag

SEQ ID NO: 51 - DNA, 23 bp, Homo sapiens
ataatggtgtgcctggcaactag

SEQ ID NO: 52 - DNA, 23 bp, Homo sapiens
acactggcctgcctgggcactag

SEQ ID NO: 53 - DNA, 23 bp, Homo sapiens
agcgcgggctcctccttgacggg

SEQ ID NO: 54 - DNA, 23 bp, Homo sapiens
agggcctgctgctccttcagcag

SEQ ID NO: 55 - DNA, 23 bp, Homo sapiens
agggctgacagctccttgagtgg

SEQ ID NO: 56 - DNA, 23 bp, Homo sapiens
ggggtgggctgctcctggagcag

SEQ ID NO: 57 - DNA, 23 bp, Homo sapiens
agagcggcctgctcctcgagggg

SEQ ID NO: 58 - DNA, 23 bp, Homo sapiens
ggggtgggctgcaccttgagggg

SEQ ID NO: 59 - DNA, 23 bp, Homo sapiens
aaggcaggctcctccttgagagg

SEQ ID NO: 60 - DNA, 23 bp, Homo sapiens
aggaagggctgctctttgaggag

SEQ ID NO: 61 - DNA, 23 bp, Homo sapiens
aggctgggctgctctttgagcag

SEQ ID NO: 62 - DNA, 23 bp, Homo sapiens
agtgccggctgctcctggagtgg

SEQ ID NO: 63 - DNA, 23 bp, Homo sapiens
agggtggggtgctcctcgagggg

SEQ ID NO: 64 - DNA, 23 bp, Homo sapiens
tgggctggctgcaccttgagtag

SEQ ID NO: 65 - DNA, 23 bp, Homo sapiens
tgggcgggctgttccttggggag

SEQ ID NO: 66 - DNA, 23 bp, Homo sapiens
cttcctgagcagccgtctgcagg

SEQ ID NO: 67 - DNA, 23 bp, Homo sapiens
ctgcctgagcagctgccacaagg

SEQ ID NO: 68 - DNA, 23 bp, Homo sapiens
cagcgttagcagccgcctgaggg

SEQ ID NO: 69 - DNA, 23 bp, Homo sapiens
cacccagagcagccgcctgacag

SEQ ID NO: 70 - DNA, 23 bp, Homo sapiens
ctgcctgggaagccgcctgccag

SEQ ID NO: 71 - DNA, 23 bp, Homo sapiens
ctgcctcctcagccgcctgaggg

SEQ ID NO: 72 - DNA, 23 bp, Homo sapiens
ctgtctgaccagccgcctgccgg

SEQ ID NO: 73 - DNA, 23 bp, Homo sapiens
cagcctgagctgccgcctgcggg

SEQ ID NO: 74 - DNA, 23 bp, Homo sapiens
caacctgagcagcctcctgagag

SEQ ID NO: 75 - DNA, 23 bp, Homo sapiens
ctccctgatcagccgcatgaggg

SEQ ID NO: 76 - DNA, 23 bp, Homo sapiens
cggccggagcagccgcctcaggg

SEQ ID NO: 77 - DNA, 23 bp, Homo sapiens
ctgcctcaacatccgcctgaaag

SEQ ID NO: 78 - DNA, 23 bp, Homo sapiens
gttgggattctgccagaaggcag

SEQ ID NO: 79 - DNA, 23 bp, Homo sapiens
gaggggggcctgccagaaggagg

SEQ ID NO: 80 - DNA, 23 bp, Homo sapiens
gcggaagatctgccagaaggggg

SEQ ID NO: 81 - DNA, 23 bp, Homo sapiens
ggtggggttctgccaggaggagg

SEQ ID NO: 82 - DNA, 23 bp, Homo sapiens
gcgggggatgtgccaggaggagg

SEQ ID NO: 83 - DNA, 23 bp, Homo sapiens
gaggggattctgccagcaggcgg

SEQ ID NO: 84 - DNA, 23 bp, Homo sapiens
gagggggtcctgccagcagggag

SEQ ID NO: 85 - DNA, 23 bp, Homo sapiens
gagggtgttctgccagcaggcag

SEQ ID NO: 86 - DNA, 23 bp, Homo sapiens
gcaggggttcagccaggaggcag

SEQ ID NO: 87 - DNA, 23 bp, Homo sapiens
gagggggttcagacagaaggcag

SEQ ID NO: 88 - DNA, 23 bp, Homo sapiens
gcaggggttctcccagtaggcag

SEQ ID NO: 89 - DNA, 23 bp, Homo sapiens
gtgggggttctgccagcagctgg

SEQ ID NO: 90 - DNA, 930 bp, Homo sapiens
atgggaatcaggctcctctgtcgtgtggccttttgtttcctggctgtaggcctcgtagatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttatgcacagcgcagcacagccgcatcctctgttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctga

SEQ ID NO: 91 - DNA, 945 bp, Homo sapiens
atgactatcaggctcctctgctacgtgggcttttattttctgggggcaggcctcatggaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtgaatggctcgggggccgtgaccaagagacccagtacttcgggccaggcacgcggctcctggtgctcgaggacctgaaaaacgtgttcccacccaaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 92 - RNA, 11 nt, Artificial Sequence
  Description of Artificial Sequence Synthetic oligonucleotide
aaaaaaaaaaa

SEQ ID NO: 93 - DNA, 945 bp, Artificial Sequence
  Description of Artificial Sequence Synthetic polynucleotide
  modified_base (4)..(6): a, c, t, g, unknown or other
  modified_base (21)..(24): a, c, t, g, unknown or other
  modified_base (29)..(29): a, c, t, g, unknown or other
  modified_base (35)..(35): a, c, t, g, unknown or other
  modified_base (39)..(39): a, c, t, g, unknown or other
  modified_base (44)..(45): a, c, t, g, unknown or other
  modified_base (47)..(47): a, c, t, g, unknown or other
  modified_base (55)..(55): a, c, t, g, unknown or other
  modified_base (57)..(57): a, c, t, g, unknown or other
  modified_base (60)..(60): a, c, t, g, unknown or other
  modified_base (62)..(64): a, c, t, g, unknown or other
  modified_base (66)..(67): a, c, t, g, unknown or other
  modified_base (69)..(71): a, c, t, g, unknown or other
  modified_base (77)..(77): a, c, t, g, unknown or other
  modified_base (79)..(79): a, c, t, g, unknown or other
  modified_base (81)..(81): a, c, t, g, unknown or other
  modified_base (87)..(87): a, c, t, g, unknown or other
  modified_base (90)..(90): a, c, t, g, unknown or other
  modified_base (93)..(93): a, c, t, g, unknown or other
  modified_base (95)..(95): a, c, t, g, unknown or other
  modified_base (97)..(97): a, c, t, g, unknown or other
  modified_base (102)..(102): a, c, t, g, unknown or other
  modified_base (106)..(106): a, c, t, g, unknown or other
  modified_base (111)..(112): a, c, t, g, unknown or other
  modified_base (114)..(116): a, c, t, g, unknown or other
  modified_base (127)..(129): a, c, t, g, unknown or other
  modified_base (132)..(135): a, c, t, g, unknown or other
  modified_base (140)..(140): a, c, t, g, unknown or other
  modified_base (147)..(147): a, c, t, g, unknown or other
  modified_base (150)..(150): a, c, t, g, unknown or other
  modified_base (155)..(155): a, c, t, g, unknown or other
  modified_base (164)..(164): a, c, t, g, unknown or other
  modified_base (171)..(171): a, c, t, g, unknown or other
  modified_base (177)..(178): a, c, t, g, unknown or other
  modified_base (182)..(183): a, c, t, g, unknown or other
  modified_base (188)..(189): a, c, t, g, unknown or other
  modified_base (192)..(192): a, c, t, g, unknown or other
  modified_base (196)..(196): a, c, t, g, unknown or other
  modified_base (198)..(198): a, c, t, g, unknown or other
  modified_base (200)..(201): a, c, t, g, unknown or other
  modified_base (204)..(204): a, c, t, g, unknown or other
  modified_base (209)..(210): a, c, t, g, unknown or other
  modified_base (216)..(219): a, c, t, g, unknown or other
  modified_base (221)..(221): a, c, t, g, unknown or other
  modified_base (225)..(225): a, c, t, g, unknown or other
  modified_base (228)..(228): a, c, t, g, unknown or other
  modified_base (235)..(235): a, c, t, g, unknown or other
  modified_base (238)..(238): a, c, t, g, unknown or other
  modified_base (240)..(243): a, c, t, g, unknown or other
  modified_base (245)..(245): a, c, t, g, unknown or other
  modified_base (248)..(249): a, c, t, g, unknown or other
  modified_base (251)..(252): a, c, t, g, unknown or other
  modified_base (258)..(258): a, c, t, g, unknown or other
  modified_base (262)..(264): a, c, t, g, unknown or other
  modified_base (266)..(266): a, c, t, g, unknown or other
  modified_base (269)..(269): a, c, t, g, unknown or other
  modified_base (275)..(276): a, c, t, g, unknown or other
  modified_base (279)..(280): a, c, t, g, unknown or other
  modified_base (287)..(288): a, c, t, g, unknown or other
  modified_base (297)..(297): a, c, t, g, unknown or other
  modified_base (303)..(304): a, c, t, g, unknown or other
  modified_base (307)..(309): a, c, t, g, unknown or other
  modified_base (312)..(312): a, c, t, g, unknown or other
  modified_base (315)..(315): a, c, t, g, unknown or other
  modified_base (319)..(320): a, c, t, g, unknown or other
  modified_base (340)..(341): a, c, t, g, unknown or other
  modified_base (345)..(348): a, c, t, g, unknown or other
  modified_base (350)..(350): a, c, t, g, unknown or other
  modified_base (363)..(364): a, c, t, g, unknown or other
  modified_base (366)..(366): a, c, t, g, unknown or other
  modified_base (368)..(368): a, c, t, g, unknown or other
  modified_base (371)..(374): a, c, t, g, unknown or other
  modified_base (376)..(376): a, c, t, g, unknown or other
  modified_base (379)..(379): a, c, t, g, unknown or other
  modified_base (381)..(382): a, c, t, g, unknown or other
  modified_base (384)..(384): a, c, t, g, unknown or other
  modified_base (387)..(387): a, c, t, g, unknown or other
  modified_base (390)..(391): a, c, t, g, unknown or other
  modified_base (394)..(394): a, c, t, g, unknown or other
  modified_base (396)..(399): a, c, t, g, unknown or other
  modified_base (402)..(403): a, c, t, g, unknown or other
  modified_base (405)..(405): a, c, t, g, unknown or other
  modified_base (417)..(417): a, c, t, g, unknown or other
  modified_base (420)..(420): a, c, t, g, unknown or other
  modified_base (433)..(433): a, c, t, g, unknown or other
  modified_base (515)..(515): a, c, t, g, unknown or other
  modified_base (519)..(519): a, c, t, g, unknown or other
  modified_base (579)..(579): a, c, t, g, unknown or other
  modified_base (753)..(753): a, c, t, g, unknown or other
  modified_base (804)..(804): a, c, t, g, unknown or other
  modified_base (810)..(810): a, c, t, g, unknown or other
  modified_base (812)..(812): a, c, t, g, unknown or other
  modified_base (816)..(816): a, c, t, g, unknown or other
  modified_base (859)..(859): a, c, t, g, unknown or other
  modified_base (877)..(877): a, c, t, g, unknown or other
  modified_base (885)..(885): a, c, t, g, unknown or other
  modified_base (897)..(897): a, c, t, g, unknown or other
  modified_base (903)..(903): a, c, t, g, unknown or other
  modified_base (907)..(907): a, c, t, g, unknown or other
  modified_base (935)..(935): a, c, t, g, unknown or other
  modified_base (944)..(945): a, c, t, g, unknown or other
atgnnnatcaggctcctctgnnnngtggncttttntttnctggnngnaggcctcntngangnnnanntnnnccagancncnagatanctngtnananggacngganagaanntnnntctggaatgtnnncannnnatggnccatganaanatgtnctggtatcnacaaganccaggnntggnnctacnnctnatcnantnntcntatgnngttaannnnanaganaanggagatnttncnnnngngtnnanngtctcnagannnanganggagcnnttnnccctgannctggagtcngccagnnccnnncanacntctnngtacctctgtgccagcagtnnatgnnnngngggccgtgaccannananccnnnncntcngnncnggnacnnggntnnnngtnntngaggacctgaanaangtgttcccacccnaggtcgctgtgtttgagccatcagaagcagagatctcccacacccaaaaggccacactggtgtgcctggccacaggcttctncccngaccacgtggagctgagctggtgggtgaatgggaaggaggtgcacagtggggtcagcacngacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaaccngtcacccagatcgtcagcgccgaggcctggggtagagcagactgtggcttnacctcngngtcntaccagcaaggggtcctgtctgccaccatcctctatgagatcntgctagggaaggccaccntgtatgcngtgctggtcagngccctngtgntgatggccatggtcaagagaaaggattncagaggctnn